(19) United States
(12) Patent Application Publication
(10) Pub. No.: US 2009/0076064 A1
(43) Pub. Date: Mar. 19, 2009

(54) Compounds

(75) Inventors: Rebecca Urbanek, Wilmington, DE (US); Dean Brown, Wilmington, DE (US); Gary Steelman, Wilmington, DE (US); William Blackwell, Roxboro, NC (US); Steven Wesolowski, Wilmington, DE (US); Xia Wang, Wilmington, DE (US)

Correspondence Address: ASTRA ZENECA PHARMACEUTICALS LP;GLOBAL INTELLECTUAL PROPERTY, 1800 CONCORD PIKE, WILMINGTON, DE, 19850-5437, US

(73) Assignee: ASTRAZENECA AB, Sodertalje (SE)

(21) Appl. No.: 12/159,993

(22) PCT Filed: Jan. 3, 2007
(86) PCT No.: PCT/SE07/00003

§ 371 Date: Jul. 3, 2008

Related U.S. Application Data
(60) Provisional application No. 60/756,684, filed on Jan. 6, 2006.

(51) Int. Cl. A61K 31/439; C07D 471/08; C07D 401/12; A61K 31/4545

(52) U.S. Cl. 514/299; 546/183; 514/318; 546/194

(57) ABSTRACT

Disclosed are compounds of Formula (I) wherein G, R², A, D and E are as described in the specification, or pharmaceutically-acceptable salts, or in vivo-hydrolysable precursors thereof. Also disclosed herein is at least one method of making, at least one pharmaceutical composition containing, and at least one method for using at least one compound in accordance with Formula I.

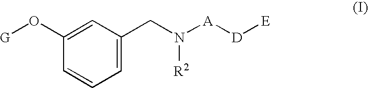

### FIELD OF THE INVENTION

Disclosed herein is at least one compound, at least one pharmaceutical composition, and at least one method useful in the treatment or prophylaxis of at least one condition or disorder related to mood changes, anxiety, depression, obesity and related disorders, eating disorders, psychiatric disorders, neurological disorders, and pain.

### BACKGROUND OF THE INVENTION

The actions of melanin-concentrating hormone (MCH) are thought to be involved in anxiety, depression, obesity and obesity-related conditions which are of growing concern, and which impact the lives of countless people. MCH is a cyclic neuropeptide involved in the regulation of a variety of brain functions. MCH induces mice to eat and MCH blockers block feeding induced by MCH; also, mice that lack the receptor for MCH are lean and do not eat when given MCH. Human and mouse MCH receptors are similar and are similarly distributed suggesting that MCH blockers will be useful for treating obesity and obesity-related disorders in humans. MCH is also thought to be involved in mood, stress and anxiety because in some animal studies MCH has induced anxiety and in others it has been axiolytic. Other studies have shown lowered activity in animals receiving MCH suggesting a depressive effect, but yet other studies have shown anti-depressive effects.

MCH has been found to be a major regulator of eating behavior and energy homeostasis and is the natural ligand for the 353-amino acid orphan G-protein-coupled-receptor (GPCR) termed SLC-1 (also known as GPR24). SLC-1 is sequentially homologous to the somatostatin receptors, and is frequently referred to as the “melanin-concentrating hormone receptor” (MCH receptor type 1, MCH1 receptor, or MCHR1).

In mice lacking the MCH1 receptor, there is no increased feeding response to MCH, and a lean phenotype is seen, suggesting that this receptor is responsible for mediating the feeding effect of MCH. MCH receptor antagonists have also been shown to block the feeding effects of MCH, and to reduce body weight & adiposity in diet-induced obese rats. The conservation of distribution and sequence of MCH1 receptors suggest a similar role for this receptor in man and rodent species. Hence, MCH receptor antagonists have been proposed as a treatment for obesity and other disorders characterised by excessive eating and body weight.

Emerging evidence also suggests that MCHR1 plays a role in the regulation of mood and stress. Within the central nervous system, MCHR1, mRNA, and protein are distributed in various hypothalamic nuclei including, for example, the paraventricular nucleus (PVN) and the nucleus accumbens shell; and limbic structures including, for example, the hippocampus, septum, amygdala, locus coeruleus and dorsal raphe nucleus, all of which are thought to be involved in the regulation of emotion and stress.

Introduction of MCH into the medial preoptic area has been reported to induce anxiety, although contrary anxiolytic-like effects of MCH injection have also been reported. Injection of MCH into the nucleus accumbens shell, in which MCHR1 is abundant, decreased mobility in a forced swim test in rats, suggesting a depressive effect. Also, it has been reported that MCHR1 antagonists exhibited antidepressant and anxiolytic-like effects in rodent tests, suggesting a role for MCHR1 in depression and anxiety.

MCH antagonists are thus thought likely to provide benefit to numerous people and to have a potential to alleviate anxiety and depression and be useful for treating obesity and obesity-related conditions.

### SUMMARY OF THE INVENTION

Described herein are compounds of Formula I:

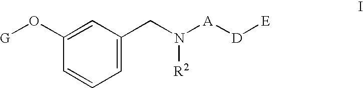

wherein:

G is selected from:

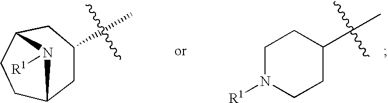

R¹ is hydrogen, —C₁₋₆alkyl, —C₁₋₆haloalkyl, —C₃₋₈cycloalkyl, or —C₃₋₈cyclooxyalkyl;

R² is hydrogen or C₁₋₄alkyl;

A is —CH₂— or —C(═O)—;

D is a 5- or 6-membered aromatic heterocyclic moiety having 1, 2 or 3 heteroatoms selected from nitrogen, oxygen, and sulfur wherein not more than one of said heteroatoms is oxygen or sulfur, or an 8-, 9- or 10-membered fused aromatic heterocyclic moiety having 1, 2 or 3 heteroatoms selected from nitrogen, oxygen or sulfur wherein not more than one of said heteroatoms is oxygen or sulfur;

E is hydrogen, halogen, —CN, —NO₂, —CF₃, —CONR⁷R⁸, —S(O)ₙR⁷, —NR⁷R⁸, —CH₂NR⁷R⁸, —OR⁷, —CH₂OR⁷, —NC(═O)R⁷, —CO₂R⁷, —C₁₋₆alkyl, —C₂₋₆alkenyl, —C₂₋₆alkynyl, C₁₋₆alkoxy, C₃₋₈cycloalkyl, —O—CH₂—O—, or -J-Ar;

J is —O—, —CH₂—, —O—CH₂— or a bond;

Ar is a 5- or 6-membered aromatic or heteroaromatic moiety having 0, 1 or 2 nitrogen atoms, 0 or 1 oxygen atoms, and 0 or 1 sulfur atoms, or is selected from an 8-, 9- or 10-membered fused aromatic or heteroaromatic ring system having 0, 1, 2 or 3 nitrogen atoms, 0 or 1 oxygen atoms, and 0 or 1 sulfur atoms;

wherein Ar is unsubstituted or has 1, 2 or 3 substituents independently selected at each occurrence from —C₁₋₆alkyl, —C₂₋₆alkenyl, —C₂₋₆alkynyl, halogen, —CN, —NO₂, —CF₃, —CONR⁷R⁸, —S(O)ₙR⁷, —NR⁷R⁸, —CH₂NR⁷R⁸, —OR⁷, —CH₂OR⁷, —NC(═O)R⁷, and —CO₂R⁷;

n is 1, 2, or 3; and

R⁷ and R⁸ are each independently hydrogen, CF₃, C₁₋₆alkyl, and/or C₃₋₈cycloalkyl;

or in vivo-hydrolysable precursors or pharmaceutically-acceptable salts thereof.

Further described herein is at least one method of treatment or prophylaxis of a disease or condition in which modulation of the MCH1 receptor is beneficial comprising administering to a patient suffering from said disease or condition a therapeutically-effective amount of at least one compound of Formula I, or in vivo-hydrolysable precursors or pharmaceutically-acceptable salts thereof.

Even further described herein is at least one method of treatment or prophylaxis of mood changes, anxiety, depression, generalized anxiety disorder, panic attacks, panic disorder, obsessive-compulsive disorder and bipolar disorders, obesity and related disorders, eating disorders, psychiatric disorders, neurological disorders and pain, comprising administering to a patient suffering therefrom a therapeutically-effective amount of at least one compound of Formula I, or in vivo-hydrolysable precursors or pharmaceutically-acceptable salts thereof.

Yet even further described herein is at least one pharmaceutical composition comprising at least one pharmaceutically-acceptable diluent, lubricant, and/or carrier and at least one compound of Formula I, or in vivo-hydrolysable precursors or pharmaceutically-acceptable salts thereof.

Still further described herein is the use of at least one compound in accord with Formula I, or in vivo-hydrolysable precursors or pharmaceutically-acceptable salts thereof, for the treatment or prophylaxis of at least one disease or condition in which modulation of the MCH1 receptor is beneficial

Still even further described herein is the use in the manufacture of a medicament for the treatment or prophylaxis of at least one disease or condition in which modulation of the MCH1 receptor is beneficial of at least one compound in accord with Formula I, or in vivo-hydrolysable precursors or pharmaceutically-acceptable salts thereof.

### DETAILED DESCRIPTION OF THE INVENTION

The features and advantages of the invention may be more readily understood by those of ordinary skill in the art upon reading the following detailed description. It is to be appreciated that certain features of the invention that are, for clarity reasons, described above and below in the context of separate embodiments, may also be combined to form a single embodiment. Conversely, various features of the invention that are, for brevity reasons, described in the context of a single embodiment, may also be combined so as to form sub-combinations thereof.

Embodiments identified herein as exemplary or preferred are intended to be illustrative and not limiting.

Unless specifically stated otherwise herein, references made in the singular may also include the plural. For example, “a” and “an” may refer to either one, or one or more.

Unless otherwise indicated, any heteroatom with unsatisfied valences is assumed to have hydrogen atoms sufficient to satisfy the valences.

The definitions set forth herein take precedence over definitions set forth in any patent, patent application, and/or patent application publication incorporated herein by reference.

Disclosed herein are compounds of Formula I:

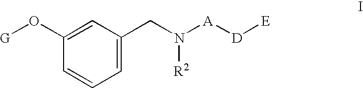

wherein:

G is:

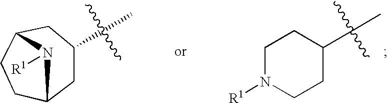

R¹ is H, —C₁₋₆alkyl, C₁₋₆haloalkyl, —C₃₋₈cycloalkyl, or —C₃₋₈cyclooxyalkyl;

R² is hydrogen or —C₁₋₄alkyl;

A is —CH₂— or —C(═O)—,

D is a 5- or 6-membered aromatic heterocyclic moiety having 1, 2 or 3 heteroatoms selected from nitrogen, oxygen or sulfur where not more than one of said heteroatoms is oxygen or sulfur, or an 8-, 9- or 10-membered fused aromatic heterocyclic moiety having 1, 2 or 3 heteroatoms selected from nitrogen, oxygen or sulfur where not more than one of said heteroatoms is oxygen or sulfur;

E is a hydrogen, halogen, —CN, —NO₂, —CF₃, —CONR⁷R⁸, —S(O)ₙR⁷, —NR⁷R⁸, —CH₂NR⁷R⁸, —OR⁷, —CH₂OR⁷, —NC(═O)R⁷, —CO₂R⁷, —C₁₋₆alkyl, —C₂₋₆alkenyl, —C₂₋₆alkynyl, C₁₋₆alkoxy, C₃₋₈cycloalkyl, —O—CH₂—O—, or -J-Ar;

J is —O—, —CH₂—, —O—CH₂— or a bond;

Ar is a 5- or 6-membered aromatic or heteroaromatic moiety having 0, 1 or 2 nitrogen atoms, 0 or 1 oxygen atoms, and 0 or 1 sulfur atoms, or an 8-, 9- or 10-membered fused aromatic or heteroaromatic ring system having 0, 1, 2 or 3 nitrogen atoms, 0 or 1 oxygen atoms, and 0 or 1 sulfur atoms;

wherein Ar is unsubstituted or has 1, 2 or 3 substituents independently selected at each occurrence from —C₁₋₆alkyl, —C₂₋₆alkenyl, —C₂₋₆alkynyl, halogen, —CN, —NO₂, —CF₃, —CONR⁷R⁸, —S(O)ₙR⁷, —NR⁷R⁸, —CH₂NR⁷R⁸, —OR⁷, —CH₂OR⁷, —NC(═O)R⁷, and —CO₂R⁷;

n is 1, 2, or 3; and

R⁷ and R⁸ are each independently hydrogen, CF₃, C₁₋₆alkyl and/or C₃₋₈cycloalkyl; or in vivo-hydrolysable precursors or pharmaceutically-acceptable salts thereof.

Particular compounds disclosed herein are those in accord with Formula I:

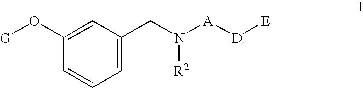

wherein:

A is —C(═O)— and G, R¹, R², D and E are as heretofore defined; or in vivo-hydrolysable precursors or pharmaceutically-acceptable salts thereof.

Other particular compounds disclosed herein are those wherein:

R¹ is H or C₁₋₄alkyl;

D is a moiety of formula II, III, IV, V, VI, or VII:

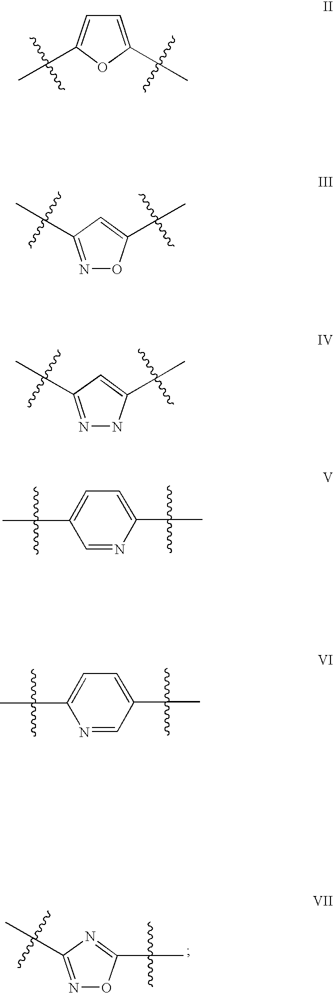

E is -J-Ar or halogen;

J is —O— or a bond; and

Ar is as heretofore defined;

or in vivo-hydrolysable precursors or pharmaceutically-acceptable salts thereof.

Other particular compounds disclose herein are those in accord with Formula I:

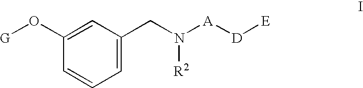

wherein:

G is:

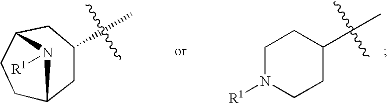

R¹ is H or methyl;

R² is hydrogen or methyl;

A is —C(═O)—;

D is a moiety of formula III, IV, V, VI, or VII:

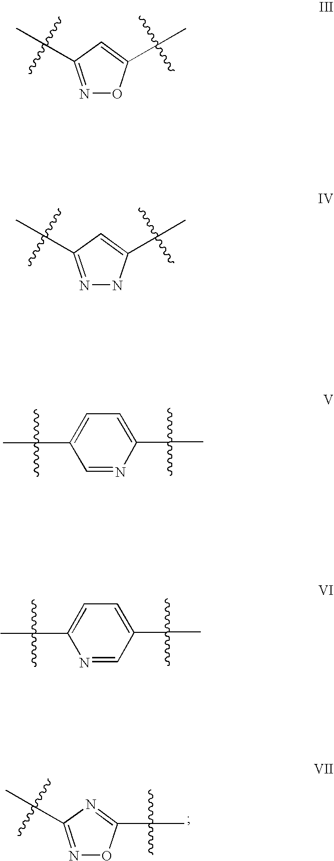

E is J-Ar;

J is a bond; and

Ar is a phenyl, phenoxy or phenyl substituted with —Cl or —O—CH₃; or

in vivo-hydrolysable precursors or pharmaceutically-acceptable salts thereof.

Yet even further disclosed herein are compounds according to Formula I including, but not limited to, for example, example compounds 1 to 30 as set forth in the following table:

| Ex | |
|---|---|
| # | Structure |
| 1. | 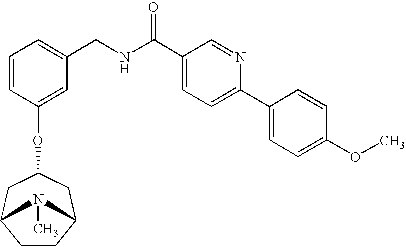 |
| 2. | 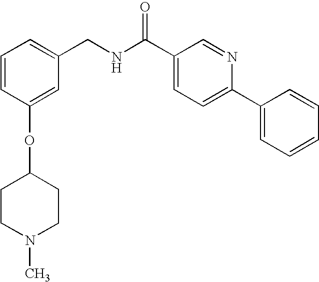 |
| 3. | 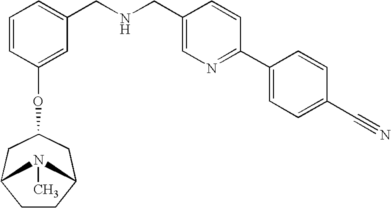 |
| 4. | 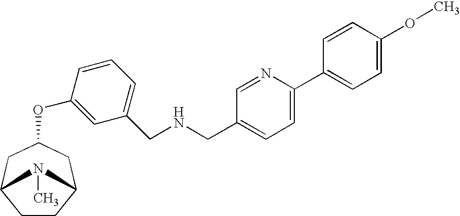 |
| 5. | 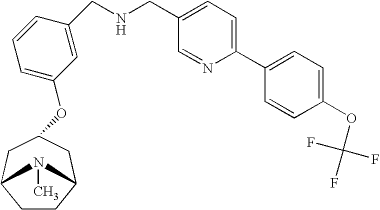 |
| 6. | 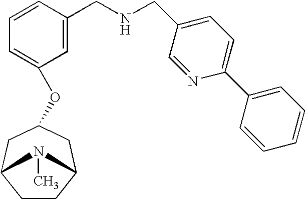 |
| 7. | 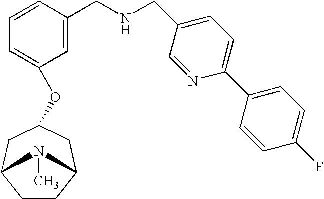 |
| 8. | 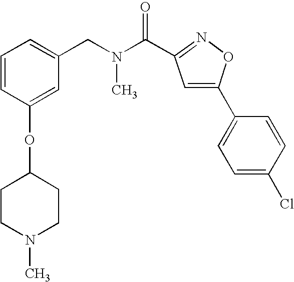 |
| 9. | 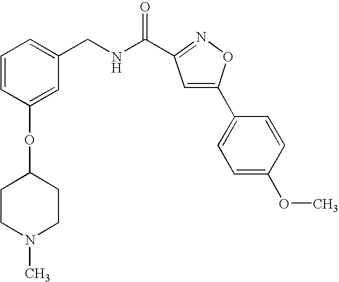 |
| 10. | 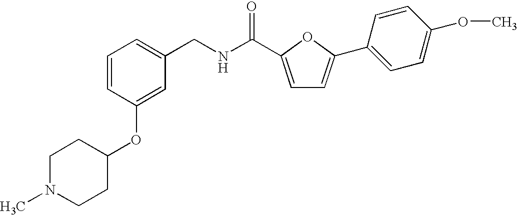 |
| 11. | 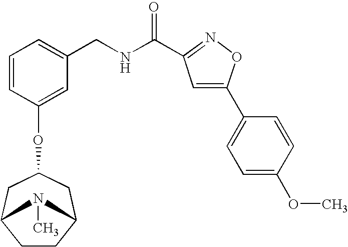 |
| 12. | 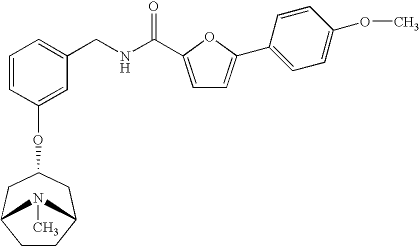 |
| 13. | 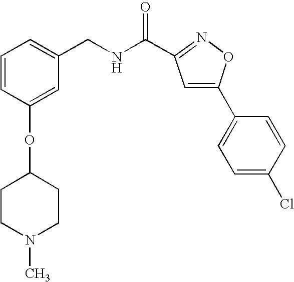 |
| 14. | 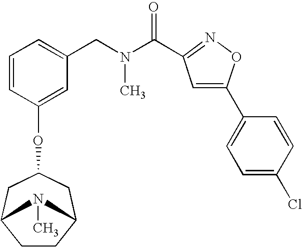 |
| 15. | 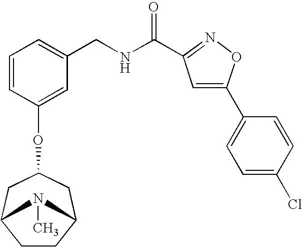 |
| 16. | 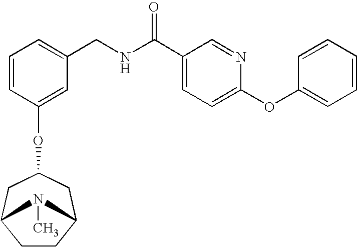 |
| 17. | 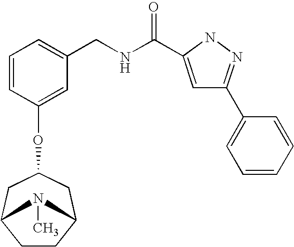 |
| 18. | 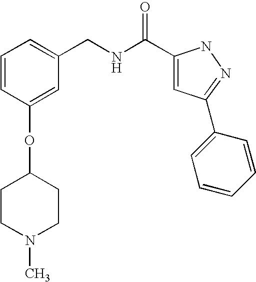 |
| 19. | 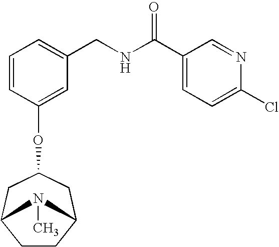 |
| 20. | 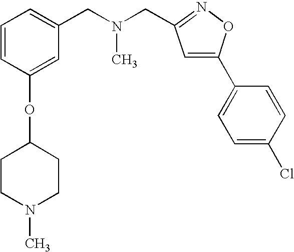 |
| 21. | 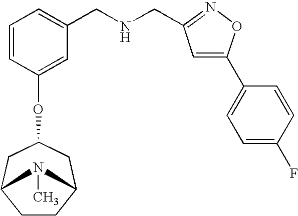 |
| 22. | 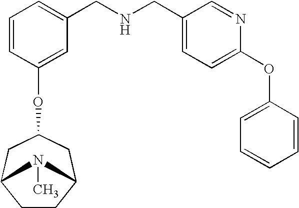 |
| 23. | 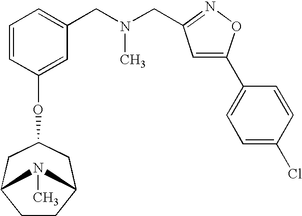 |
| 24. | 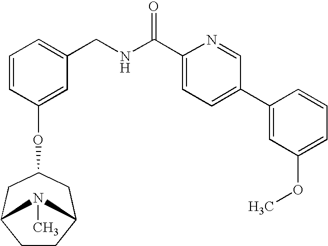 |
| 25. | 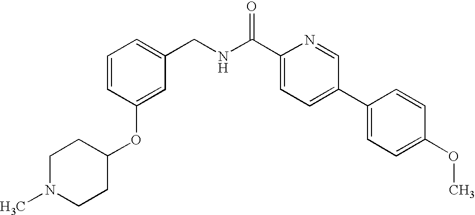 |
| 26. | 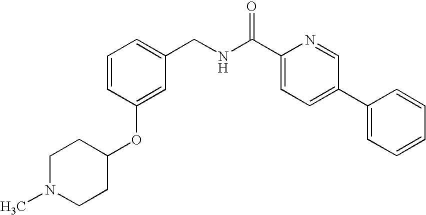 |
| 27. | 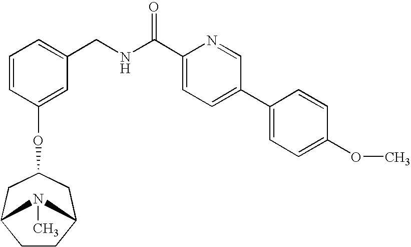 |
| 28. | 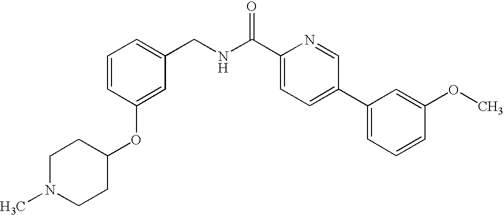 |
| 29. | 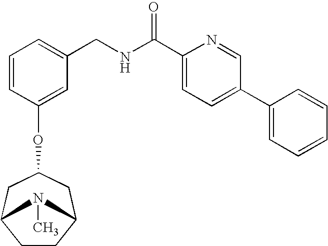 |
| 30. | 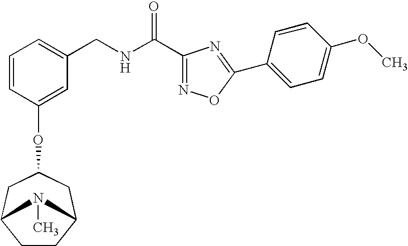 |

Further compounds disclosed herein are in accord with Formula I:

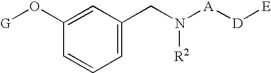

wherein:

G is selected from:

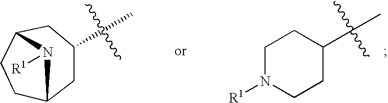

R¹ is selected from H, —C₁₋₆alkyl, C₁₋₆haloalkyl, —C₃₋₈cycloalkyl and —C₃₋₈cyclooxyalkyl;

R² is selected from hydrogen or —C₁₋₄alkyl;

A is selected from —CH₂— or —C(═O)—;

D is a 5- or 6-membered aromatic heterocyclic moiety having 1, 2 or 3 heteroatoms selected from nitrogen, oxygen or sulfur where not more than one of said heteroatoms is oxygen or sulfur, or D is an 8-, 9- or 10-membered fused aromatic heterocyclic moiety having 1, 2 or 3 heteroatoms selected from nitrogen, oxygen or sulfur where not more than one of said heteroatoms is oxygen or sulfur;

E is selected from hydrogen, halogen, —CN, —NO₂, —CF₃, —CONR⁷R⁸, —S(O)R⁷, —NR⁷R⁸, —CH₂NR⁷R⁸, —OR⁷, —CH₂OR⁷, —NC(═O)R⁷, —CO₂R⁷, —C₁₋₆alkyl, —C₂₋₆alkenyl, —C₂₋₆alkynyl, C₁₋₆alkoxy, C₃₋₈cycloalkyl, —O—CH₂—O—, or -J-Ar;

wherein J is —O—, —CH₂—, —O—CH₂— or a bond, and Ar is selected from a 5- or 6-membered aromatic or heteroaromatic moiety having 0, 1 or 2 nitrogen atoms, 0 or 1 oxygen atoms, and 0 or 1 sulfur atoms, or is selected from an 8-, 9- or 10-membered fused aromatic or heteroaromatic ring system having 0, 1, 2 or 3 nitrogen atoms, 0 or 1 oxygen atoms, and 0 or 1 sulfur atoms;

wherein Ar is unsubstituted or has 1, 2 or 3 substituents independently selected at each occurrence from —C₁₋₆alkyl, —C₂₋₆alkenyl, —C₂₋₆alkynyl, halogen, —CN, —NO₂, —CF₃, —CONR⁷R⁸, —S(O)ₙR⁷, —NR⁷R⁸, —CH₂NR⁷R⁸, —OR⁷, —CH₂OR⁷, —NC(═O)R⁷ or —CO₂R⁷;

wherein n is 1, 2, or 3; and

wherein R⁷ and R⁸ are independently selected from hydrogen, C₁₋₆alkyl or C₃₋₈cycloalkyl; or in vivo-hydrolysable precursors or pharmaceutically-acceptable salts thereof.

Even further disclosed herein are compounds in accord with Formula I:

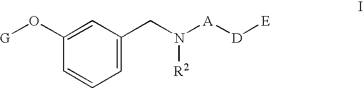

wherein:

A is —C(═O)— and G, R¹, R², D and E are as heretofore defined;

or in vivo-hydrolysable precursors or pharmaceutically-acceptable salts thereof.

Further disclosed herein are compounds in accord with Formula I:

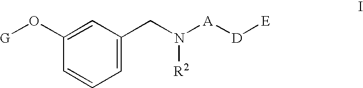

wherein:

R¹ is selected from H or C₁₋₄alkyl;

D is selected from a moiety of formula II, III, IV, V, or VI:

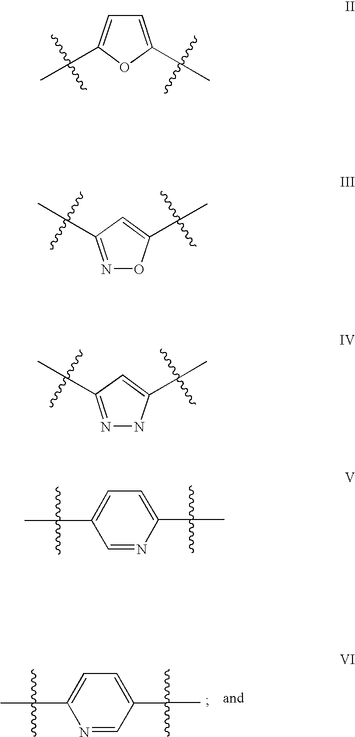

E is -J-Ar or halogen, wherein J is —O— or a bond and Ar is as heretofore defined; or in vivo-hydrolysable precursors or pharmaceutically-acceptable salts thereof.

Yet even further disclosed herein are compounds in accord with Formula I:

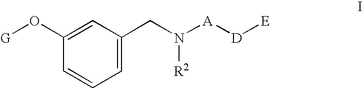

wherein:

G is selected from:

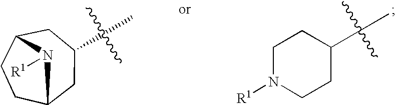

R¹ is H or methyl;

R² is hydrogen or methyl;

A is —C(═O)—;

D is a moiety of formula III, IV, V, or VI:

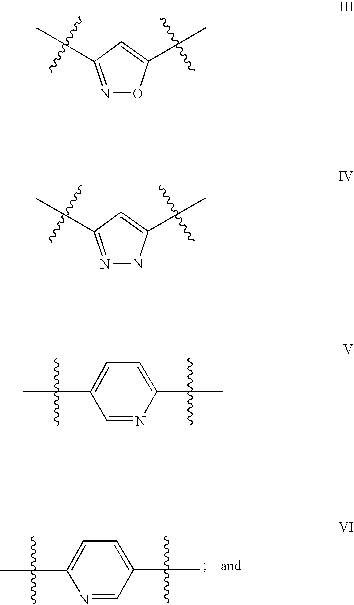

E is J-Ar, wherein J is a bond and Ar is a phenyl, phenoxy or phenyl substituted with —Cl or —O—CH₃; or in vivo-hydrolysable precursors or pharmaceutically-acceptable salts thereof.

Yet even further disclosed herein are compounds according to Formula I including, but not limited to, for example, example compounds 1 to 29 as set forth in the following table:

| Ex | |
|---|---|
| # | Structure |
| 1. | 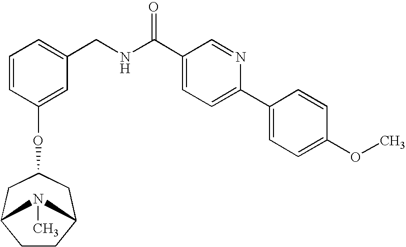 |
| 2. | 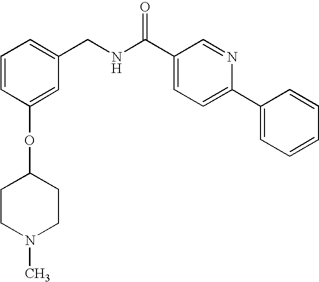 |
| 3. | 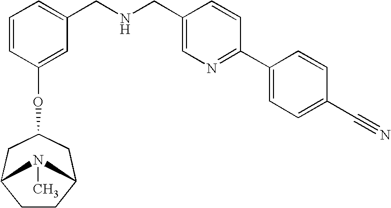 |
| 4. | 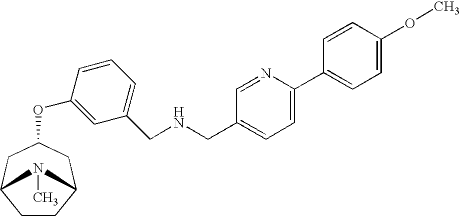 |
| 5. | 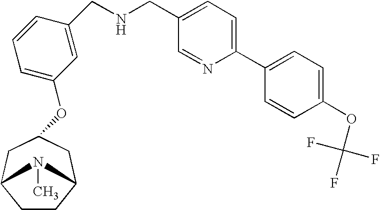 |
| 6. | 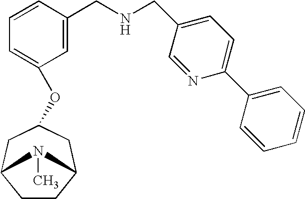 |
| 7. | 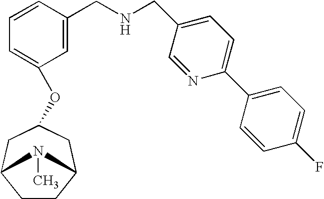 |
| 8. | 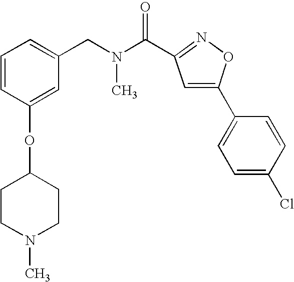 |
| 9. | 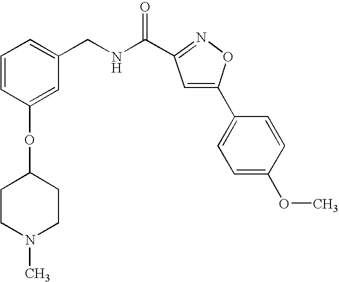 |
| 10. | 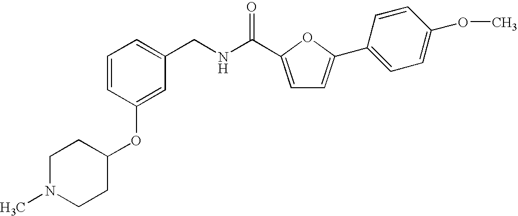 |
| 11. | 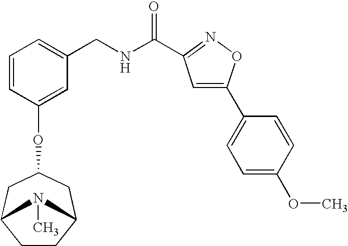 |
| 12. | 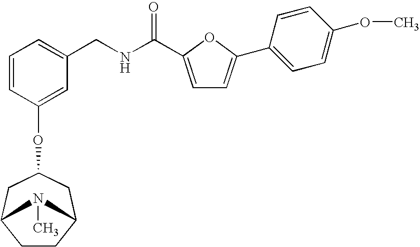 |
| 13. | 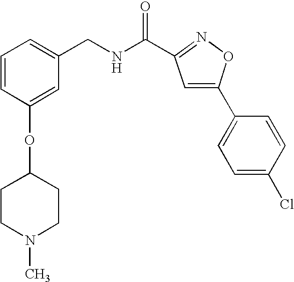 |
| 14. | 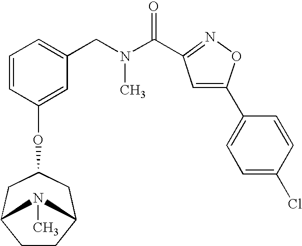 |
| 15. | 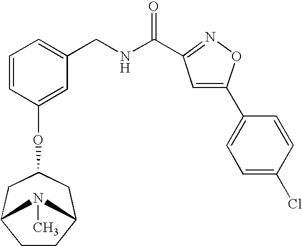 |
| 16. | 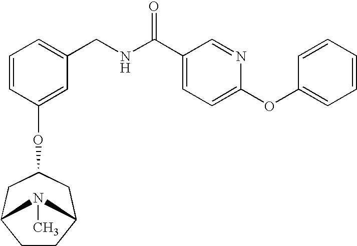 |
| 17. | 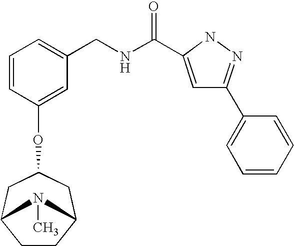 |
| 18. | 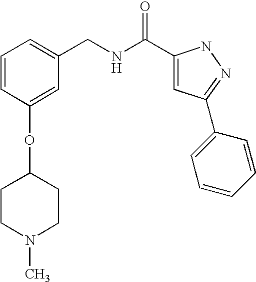 |
| 19. | 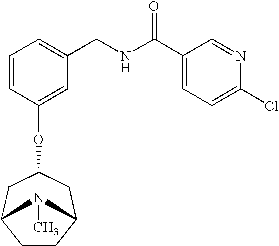 |
| 20. | 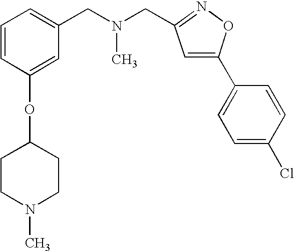 |
| 21. | 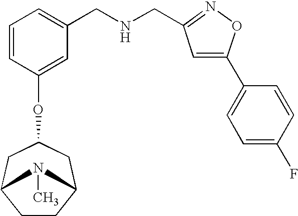 |
| 22. | 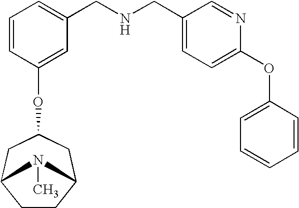 |
| 23. | 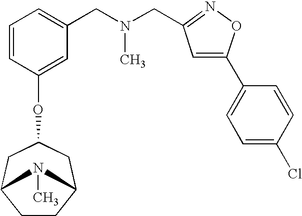 |
| 24. | 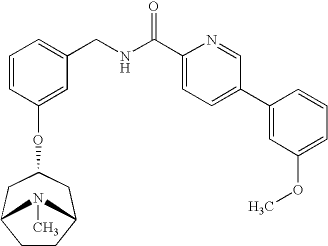 |
| 25. | 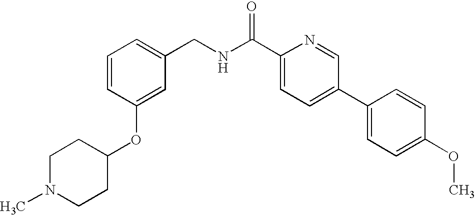 |
| 26. | 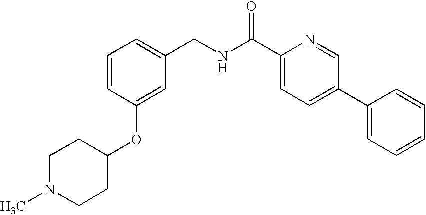 |
| 27. | 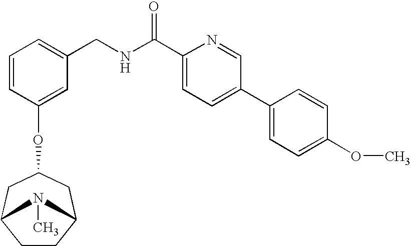 |
| 28. | 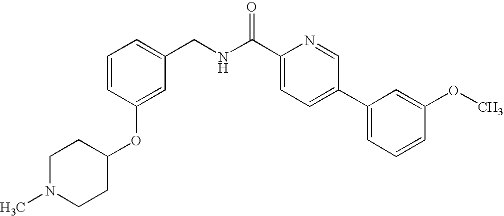 |
| 29. | 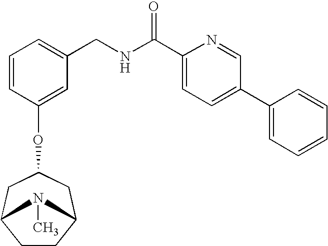 |

In one embodiment, at least one compound of Formula I is provided wherein A is —CH₂—.

In another embodiment, at least one compound of Formula I is provided wherein G is:

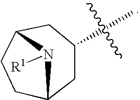

In yet another embodiment, at least one compound of Formula I is provided wherein

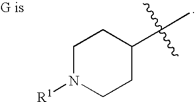

In a further embodiment, at least one compound of Formula I is provided wherein D is formula

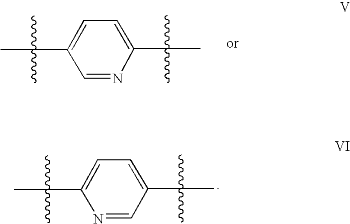

In a still further embodiment, at least one compound of Formula I is provided wherein Ar is a 5- or 6-membered aromatic or heteroaromatic moiety having 0, 1 or 2 nitrogen atoms, 0 or 1 oxygen atoms, and 0 or 1 sulfur atoms, wherein said Ar has 1, 2 or 3 substituents independently selected at each occurrence from halogen, —CN, and OR⁷.

In an even further embodiment, at least one compound of Formula I is provided wherein Ar is a 5- or 6-membered aromatic moiety having 0, 1 or 2 nitrogen atoms, 0 or 1 oxygen atoms, and 0 or 1 sulfur atoms, wherein said Ar has 1, 2 or 3 substituents independently selected at each occurrence from halogen, —CN, and OR⁷.

In yet an even further embodiment, at least one compound of Formula I is provided wherein Ar has 1, 2 or 3 substituents independently selected at each occurrence from Cl, F, —CN, OCF₃, and OCH₃.

In still yet an even further embodiment, at least one compound of Formula I is provided wherein Ar is a 5- or 6-membered aromatic or heteroaromatic moiety having 0, 1 or 2 nitrogen atoms, 0 or 1 oxygen atoms, and 0 or 1 sulfur atoms, and Ar is unsubstituted.

A further embodiment relates to compounds as described herein wherein one or more of the atoms is a radioisotope of the same element. In a particular embodiment, the compound is labeled with tritium. Such radio-labeled compounds are synthesized either by incorporating radio-labeled starting materials or, in the case of tritium, exchange of hydrogen for tritium by known methods. Known methods include (1) electrophilic halogenation, followed by reduction of the halogen in the presence of a tritium source, for example, by hydrogenation with tritium gas in the presence of a palladium catalyst, or (2) exchange of hydrogen for tritium performed in the presence of tritium gas and a suitable organometallic (e.g. palladium) catalyst.

A compound labeled with tritium may be useful in identifying novel medicinal compounds capable of binding to and modulating the activity, by agonism, partial agonism, or antagonism, of an MCH1 receptor. Such tritium-labeled compounds may be used in assays that measure the displacement of such compounds to assess the binding of ligands that bind to MCH1 receptors.

In an even further embodiment, compounds disclosed herein may additionally comprise one or more atoms of a radioisotope. In a particular form of this embodiment, a compound comprises a radioactive halogen. Such radio-labeled compounds are synthesized by incorporating radio-labeled starting materials by known methods. In a particular embodiment, the radioisotope is selected from ¹⁸F, ¹²³I, ¹²⁵I, ¹³¹I, ⁷⁵Br, ⁷⁶Br, ⁷⁷Br or ⁸²Br. In a more particular embodiment, the radioisotope is ¹⁸F.

Still yet another embodiment encompasses using compounds of Formula I for the therapy of at least one disease mediated through the action of at least one MCH1 receptor. A more particular embodiment relates to using antagonistic-compounds of Formula I, or in vivo-hydrolysable precursors or pharmaceutically-acceptable salts thereof, for the therapy of at least one disease mediated through the action of at least one MCH1 receptor.

A further embodiment encompasses a method of treatment or prophylaxis of at least one disease or condition in which modulation of the MCH1 receptor is beneficial comprising administering a therapeutically-effective amount of at least one compound of Formula I, or in vivo-hydrolysable precursors or pharmaceutically-acceptable salts thereof, to a subject suffering from said disease or condition.

An even further embodiment is directed to a method of treatment or prophylaxis of at least one disease or condition in which modulating the MCH1 receptor is beneficial comprising administering a therapeutically-effective amount of at least one antagonistic compound of Formula I, or in vivo-hydrolysable precursors or pharmaceutically-acceptable salts thereof, to a patient suffering from said disease or condition.

A further aspect of the invention is directed to the use of at least one compound of Formula I, or an enantiomer or in vivo-hydrolysable precursor and/or pharmaceutically-acceptable salt thereof, for the treatment or prophylaxis of at least one disease or condition in which modulation of at least one MCH1 receptor is beneficial.

Another embodiment is directed to a pharmaceutical composition comprising a compound in accordance with Formula I, or in vivo-hydrolysable precursors or pharmaceutically-acceptable salts thereof, and at least one pharmaceutically-acceptable diluent, lubricant, and/or carrier.

A further embodiment relates to a pharmaceutical composition useful for treatment or prophylaxis of a disease or condition mentioned herein arising from dysfunction of MCH1 receptors in a patient comprising a therapeutically-effective amount of at least one compound of Formula I, or an enantiomer or in vivo-hydrolysable precursor or pharmaceutically-acceptable salt thereof, effective in treating or preventing such disease or condition, and at least one pharmaceutically-acceptable diluent, lubricant, and/or carrier.

An even further embodiment relates to a pharmaceutical composition useful for treatment or prophylaxis of a disease or condition mentioned herein arising from dysfunction of MCH1 receptors in a patient comprising a therapeutically-effective amount of at least one antagonistic compound of Formula I, or an enantiomer or in vivo-hydrolysable precursor or pharmaceutically-acceptable salt thereof, effective in treating or preventing such disease or condition, and at least one pharmaceutically-acceptable diluent, lubricant, and/or carrier

In yet an even further embodiment, the at least one disease or condition includes, but is not limited to, for example, a disorder, such as, for example, a mood disorder, anxiety, and/or depression.

In yet an even more particular embodiment, the disorder includes, but is not limited to, for example, anxiety, generalized anxiety disorder, panic attacks, panic disorder, obsessive-compulsive disorder, depression, and/or bipolar disorders.

In an even further embodiment, the at least one disease or condition includes, but is not limited to, for example, obesity and related disorders, eating disorders, psychiatric disorders, neurological disorders, and/or pain.

In yet another embodiment, a method is provided for treating obesity, psychiatric disorders, anxiety, anxio-depressive disorders, depression, bipolar disorder, ADHD, cognitive disorders, memory disorders, schizophrenia, epilepsy, neurological disorders, pain related disorders, and/or related conditions comprising administering a pharmacologically effective amount of at least one compound of Formula I, or in vivo-hydrolysable precursors or pharmaceutically-acceptable salts thereof, to a patient in need thereof.

Still another embodiment provides compounds useful for treating obesity, type II diabetes, metabolic syndrome, and/or preventing type II diabetes comprising administering a pharmacologically effective amount of at least one compound of Formula I, or in vivo-hydrolysable precursors or pharmaceutically-acceptable salts thereof, to a patient in need thereof.

In one embodiment, the patient/subject is an animal.

In another embodiment, the patient/subject is a mammalian species including, but not limited to, for example, humans and domestic animals, such as, for example, dogs, cats, and horses.

In yet a further embodiment, the patient/subject is a human.

Yet another embodiment provides at least one process for preparing at least one compound of Formula I.

A further embodiment is directed to the use of at least one compound of Formula I, an enantiomer thereof or a pharmaceutically-acceptable salt thereof, in the manufacture of a medicament for the treatment or prophylaxis of at least one disease or condition mentioned herein.

A particular embodiment is directed to using at least one compound of Formula I, or in vivo-hydrolysable precursors or pharmaceutically-acceptable salts thereof, in the manufacture of a medicament for treatment or prophylaxis of a mood disorders, anxiety, and/or depression. More particular embodiments encompass use of at least one compound of Formula I, or in vivo-hydrolysable precursors or pharmaceutically-acceptable salts thereof, in the manufacture of a medicament for the treatment or prophylaxis of anxiety, generalized anxiety disorder, panic attacks, panic disorder, obsessive-compulsive disorder, depression, and/or bipolar disorders. Yet another embodiment provides use of at least one compound of Formula I, or in vivo-hydrolysable precursors or pharmaceutically-acceptable salts thereof, in the manufacture of a medicament for the treatment of obesity and related disorders, eating disorders, psychiatric disorders, neurological disorders, and/or pain.

For the uses, methods, medicaments and compositions mentioned herein the amount of compound used and the dosage administered may vary with the Formula I compound employed; and/or the desired mode of administration and/or treatment. However, in general, satisfactory results are obtained when a compound in accordance with Formula I is administered at a daily dosage of about 0.1 mg to about 20 mg/kg of animal body weight. Such doses may be given in divided doses 1 to 4 times a day or in a sustained release form. For man, the total daily dose is in the range of from about 5 mg to 1,400 mg, and more particularly from about 10 mg to 100 mg. Unit dosage forms suitable for oral administration generally comprise, for example, from about 2 mg to 1,400 mg of a compound in accordance with Formula I, or in vivo-hydrolysable precursors or pharmaceutically-acceptable salts thereof, admixed with at least one solid and/or liquid pharmaceutical carrier, lubricant, and/or diluent.

A compound of Formula I, enantiomers, in vivo-hydrolysable precursors, and/or pharmaceutically-acceptable salts thereof, may be used on their own or in the form of appropriate medicinal preparations for enteral or parenteral administration.

A further embodiment provides a pharmaceutical composition comprising less than 80% by weight of at least one compound according to Formula I in admixture with at least one inert pharmaceutically-acceptable diluent, lubricant, and/or carrier.

Yet a further embodiment provides a pharmaceutical composition comprising less than 50% by weight of at least one compound according to Formula I, or in vivo-hydrolysable precursors or pharmaceutically-acceptable salts thereof, in admixture with at least one inert pharmaceutically-acceptable diluent, lubricant, and/or carrier.

Exemplary diluents, lubricants, and/or carriers for tablets and dragees include, but are not limited to, for example, lactose, starch, talc, and stearic acid.

Exemplary diluents, lubricants, and/or carriers for capsules include, but are not limited to, for example, tartaric acid and lactose.

Exemplary diluents, lubricants, and/or carriers for injectable solutions include, but are not limited to, for example, water, alcohol, glycerin, and vegetable oil.

Exemplary diluents, lubricants, and/or carriers for suppositories include, but are not limited to, for example, natural or hardened oils and waxes.

Also provided herein is a process for preparing a pharmaceutical composition comprising mixing or compounding the ingredients together and forming the mixed ingredients into tablets or suppositories; encapsulating the ingredients in capsules; or dissolving the ingredients to form injectable solutions.

Tautomeric, enantiomeric, stereoisomeric and/or geometric isomers of Formula I are also contemplated herein. The various optical isomers may be isolated by separation of a racemic mixture of the compounds using conventional techniques, e.g. fractional crystallization, chiral HPLC, or chiral SFC. Alternatively the individual enantiomers may be made by reacting the appropriate optically active starting materials under reaction conditions that do not cause racemization.

Pharmaceutically-acceptable derivatives include solvates and salts. For example, the compounds of the invention can form acid addition salts with acids, such as conventional pharmaceutically-acceptable acids, including, but not limited to, for example, maleic, hydrochloric, hydrobromic, phosphoric, acetic, fumaric, salicylic, citric, lactic, mandelic, tartaric, and methanesulfonic acids.

In another embodiment, a compound of Formula I or a pharmaceutically acceptable salt, solvate or in vivo hydrolysable ester thereof, or a pharmaceutical composition or formulation comprising a compound of Formula (I) may be administered concurrently, simultaneously, sequentially, or separately with at least one other pharmaceutically active compound selected from the following:
(i) antidepressants including, but not limited to, for example, amitriptyline; amoxapine; bupropion; citalopram; clomipramine; desipramine; doxepin; duloxetine; elzasonan; escitalopram; fluvoxamine; fluoxetine; gepirone; imipramine; ipsapirone; maprotiline; nortriptyline; nefazodone; paroxetine; phenelzine; protriptyline; reboxetine; robalzotan; sertraline; sibutramine; thionisoxetine; tranylcypromaine; trazodone; trimipramine; venlafaxine; and equivalents and pharmaceutically active isomer(s) and metabolite(s) thereof.
(ii) atypical antipsychotics including, but not limited to, for example, dibenzepines, such as, for example, clozapine, loxapine, olanzapine, and quetiapine; benzisoxazoles, such as, for example, risperidone and ziprasidone; quinolinones, such as, for example, aripiprazole; and pharmaceutically active isomer(s) and metabolite(s) thereof.
(iii) antipsychotics including, but not limited to, for example, amisulpride; asenapine; benzisoxidil; bifeprunox; carbamazepine; chlorpromazine; debenzapine; divalproex; duloxetine; eszopiclone; haloperidol; iloperidone; lamotrigine; mesoridazine; paliperidone; perlapine; perphenazine; phenothiazine; phenylbutlypiperidine; pimozide; prochlorperazine; sertindole; sulpiride; suproclone; suriclone; thioridazine; trifluoperazine; trimetozine; valproate; valproic acid; zopiclone; zotepine; and equivalents and pharmaceutically active isomer(s) and metabolite(s) thereof.
(iv) anxiolytics including, but not limited to, for example, alnespirone; azaspirones, such as, for example, buspirone; benzodiazepines, such as, for example, adinazolam, alprazolam, balezepam, bentazepam, bromazepam, brotizolam, clonazepam, clorazepate, chlordiazepoxide, cyprazepam, diazepam, estazolam, fenobam, flunitrazepam, flurazepam, fosazepam, halazepam, lorazepam, lormetazepam, midazolam, nitrazepam, oxazepam, prazepam, quazepam, reclazepam, tracazolate, trepipam, temazepam, triazolam, uldazepam, and zolazepam; barbiturates, such as, for example, mephobarbital, phenobarbital, secobarbital, pentobarbital, amobarbital, and butabarbital; ethanolamines, such as, for example, diphenhydramine; carbamate derivatives, such as, for example, meprobamate; and equivalents and pharmaceutically active isomer(s) and metabolite(s) thereof;
(v) anticonvulsants, including, but not limited to, for example, acetazolamide; carbamazepine; felbamate; valproate; valproic acid; divalproex; primidone; lamotrogine; oxcarbazepine; tiagabine; topiramate; pregabalin; levetiracetam; phenyloin; zonisamide; ethosuximde; methsuximiide; gabapentin; and equivalents and pharmaceutically active isomer(s) and metabolite(s) thereof.
(vi) Alzheimer's therapies including, but not limited to, for example, donepezil; memantine; tacrine; and equivalents and pharmaceutically active isomer(s) and metabolite(s) thereof.
(vii) Parkinson's therapies including, but not limited to, for example, deprenyl; levodopa; carbidopa; entacapone; non-ergot dopamine receptor agonists, such as, for example, ropinirole, pramipexole, and apomorphine; MAO inhibitors, such as, for example, selegiline and rasagiline; comP inhibitors, such as, for example, tolcapone; A-2 inhibitors; dopamine reuptake inhibitors; NMDA antagonists; nicotine agonists; dopamine receptor agonists, such as, for example, bromocriptine mesylate; anticholinergics, such as, for example, belladonna alkaloids, benztropine, biperiden, procyclidine, and trihexyphenidyl; inhibitors of neuronal nitric oxide synthase; and equivalents and pharmaceutically active isomer(s) and metabolite(s) thereof.
(viii) migraine therapies including, but not limited to, for example, ergotamine derivatives, such as, for example, dihydroergotamine mesylate and ergotamine tartrate; serotonin 5-HT 1 receptor agonists, such as, for example, almotriptan, eletriptan, frovatriptan, naratriptan, rizatriptan, sumatriptan, and zolmitriptan; caffeine; beta-adrenergic blocking agents, such as, for example, atenolol, timolol, propranolol, and guanfacine; bromocriptine; cabergoline; dichloralphenazone; lisuride; pergolide; pramipexole; ropinirole; and equivalents and pharmaceutically active isomer(s) and metabolite(s) thereof.
(ix) stroke therapies including, but not limited to, for example, abciximab, activase, citicoline, crobenetine, desmoteplase, repinotan, traxoprodil, and equivalents and pharmaceutically active isomer(s) and metabolite(s) thereof.
(x) urinary incontinence therapies including, but not limited to, for example, darifenacin, falvoxate, oxybutynin, propiverine, robalzotan, solifenacin, tolterodine, trospium, and equivalents and pharmaceutically active isomer(s) and metabolite(s) thereof.
(xi) neuropathic pain therapies including, but not limited to, for example, gabapentin, lidoderm, pregablin and equivalents and pharmaceutically active isomer(s) and metabolite(s) thereof.
(xii) nociceptive pain therapies including, but not limited to, for example, celecoxib, etoricoxib, lumiracoxib, rofecoxib, valdecoxib, diclofenac, loxoprofen, naproxen, paracetamol, and equivalents and pharmaceutically active isomer(s) and metabolite(s) thereof.
(xiii) insomnia therapies including, but not limited to, for example allobarbital, alonimid, amobarbital, benzoctamine, butabarbital, capuride, chloral, cloperidone, clorethate, dexclamol, ethchlorvynol, etomidate, glutethimide, halazepam, hydroxyzine, mecloqualone, melatonin, mephobarbital, methaqualone, midaflur, nisobamate, pentobarbital, phenobarbital, propofol, roletamide, triclofos, secobarbital, zaleplon, zolpidem, and equivalents and pharmaceutically active isomer(s) and metabolite(s) thereof.
(xiv) mood stabilizers including, but not limited to, for example, carbamazepine, divalproex, gabapentin, lamotrigine, lithium, olanzapine, quetiapine, valproate, valproic acid, verapamil, and equivalents and pharmaceutically active isomer(s) and metabolite(s) thereof.

Any such combination products would most likely employ the compounds of this invention within the dosage range described herein and any such other pharmaceutically active compound within the approved dosage range for such other pharmaceutically active compound.

### Assay Methods:

### MCH Binding Assay:

Binding of Melanin Concentrating Hormone (MCH) may be measured with a radioligand-binding assay employing [¹²⁵I]MCH and membranes expressing human Melanin Concentrating Hormone receptor 1 (MCHR1). Ligands that bind to MCHR1 may be identified by their ability to compete with the binding of [¹²⁵I]MCH.

[¹²⁵I]MCH may be purchased from Amersham BioSource (Cat #Im344-25 μCi). Membranes (3.8 mg/mL, cat#ES-370-M, batch 1346) may be prepared from CHOK1 cells expressing human MCH receptor 1 such as those obtainable from EuroScreen. Trizma, BSA, NaCl, and MgCl₂6H₂O were from Sigma. Human MCH was purchased from Bachem (0.5 mg, cat #H-1482).

Assays may be performed in BSA pretreated plates with 2 μg membranes per well. Saturation binding assays may be run in 50 mM Tris, pH 7.4, containing 3 mM MgCl₂ and 0.5 mg/mL BSA. To perform an assay, 20 μL of 2-fold serially diluted radioligand [¹²⁵I]MCH is added to wells of a shallow 96-well plate. This is followed by addition of 180 μL of assay buffer containing membranes at a final protein concentration of 15 μg/mL. The mixture is incubated at room temperature for 1 h before being filtered through a 96 well filter-bottom plate (GF/B), previously soaked in 0.1% BSA for at least 3 h. Collected membranes are washed 3 times with 300 μL/well of wash buffer (50 mM Tris, pH 7.4, containing 5 mM MgCl₂ and 50 mM NaCl), and then dried in air overnight or at 60° C. ¹²⁵I is measured by scintillation counting.

[¹²⁵I]MCH binding assays performed in the presence of test compounds, either at fixed or a series of concentrations, may be employed in a ligand competition binding assay. For dose-response assays, compounds may be 3-fold serially diluted in an assay plate to produce a range of concentrations. For single point assays, [¹²⁵I]MCH and membranes may be pre-mixed and then transferred to an assay plates with respective final membrane protein and radioligand concentrations of 20 μg/mL and 0.04 nM.

For analysis, cpm are converted to dpm, and nM radioligand concentration is calculated using vendor-provided specific radioactivity.

Saturation binding data may be analyzed using equation (1):

$$B = \frac{{B}_{max} [ {[}^{125} I ] M C H ]}{{K}_{d} + [ {[}^{125} I ] M C H ]} \qquad ( 1 )$$

where B is concentration of bound ligand, Bₘₐₓ is the maximum concentration of bound ligand, and K_{d} is the dissociation constant for ligand.

Percent inhibition (% Inh) may be calculated using equation (2):

$$\% Inh = 100 / ( 1 - \frac{( {counts}_{sample} - {counts}_{negative} )}{( {counts}_{positive} - {counts}_{negative} )} ) \qquad ( 2 )$$

IC₅₀ values may be calculated by conventional methods using non-linear squares analysis.

At least one compound within the scope of Formula (I), including at least one compound described in the examples hereinbelow, has been tested in the MCH binding assay and exhibited activity via an IC₅₀ value of less than about 100 μM. In one embodiment, at least one compound of Formula (I) showed activity in the MCH binding assay via an IC₅₀ value of less than about 10 μM.

### MCHR1 Receptor Activation Assay:

Melanin Concentrating Hormone Receptor 1 (MCHR1) is a G-protein coupled receptor that interacts with heterotrimeric G proteins containing a Gα_{i/o} subunit. Binding of MCH to MCHR1 results in the exchange of GDP for GTP on the Gα_{i/o} proteins associated with the activated receptor. This activation can be quantified by measuring the amount of a GTP analog, GTPγ³⁵S, bound to the membrane-associated receptor. GTPγ⁵S is not hydrolyzed by the intrinsic GTPase activity of a G-protein but instead forms a stable complex. Activation of MCH1 receptors may thus be quantified by measuring the amount of GTPγ³⁵S bound to membranes prepared from cells expressing such receptors. Membranes may be isolated by filtration or may be bound on SPA beads (Amersham). Bound GTPγ³⁵S may then be quantified by determining the amount of ³⁵S present. Inhibition of MCH binding by a competing ligand may thus be assessed by a decrease in the amount of GTPγ³⁵S bound to membranes in the presence of such a competing ligand.

At least one compound within the scope of Formula (I), including at least one compound described in the examples hereinbelow, has been tested in the GTPγ³⁵S binding assay and exhibited activity via an IC₅₀ value of less than about 100 μM. In one embodiment, at least one compound of Formula (I) showed activity in the GTPγ³⁵S binding assay via an IC₅₀ value of less than about 50 μM.

### ABBREVIATIONS AND DEFINITIONS

Generally terms and abbreviations used herein are intended to have their conventional meanings. For clarity, as used herein:

“alkyl”, used alone or as a suffix or prefix, means a monovalent straight or branched chain hydrocarbon radical;

“alkoxy”, used alone or as a suffix or prefix, means an oxygen-linked monovalent straight or branched chain hydrocarbon radical;

“haloalkyl”, used alone or as a suffix or prefix, means a monovalent straight or branched chain hydrocarbon radical having at least on halogen therein;

“cycloalkyl”, used alone or as a suffix or prefix, means a monovalent hydrocarbon radical having a saturated cyclic portion therein;

“cyclooxyalkyl”, used alone or as a suffix or prefix, means a monovalent hydrocarbon radical having a cyclic portion therein having an oxygen as a member of said cyclic portion

“cycloalkyloxy”, used alone or as a suffix or prefix, means an oxygen-linked monovalent hydrocarbon radical having a cyclic portion therein;

“alkenyl”, used alone or as suffix or prefix, means a monovalent straight or branched chain hydrocarbon radical having at least one C═C bond therein;

“alkynyl”, used alone or as suffix or prefix, means a monovalent straight or branched chain hydrocarbon radical having at least one C≡C bond therein;

“5- or 6-membered aromatic heterocyclic”, used alone or as suffix or prefix, means a monovalent cyclic radical having carbon, and at least one nitrogen, oxygen or sulfur ring atom having aromatic character (i.e., 4n+2 delocalized electrons);

“8-, 9- or 10-membered fused aromatic heterocyclic”, used alone or as suffix or prefix, means a monovalent bi-cyclic radical having carbon, and at least one nitrogen, oxygen or sulfur ring atom having aromatic character (i.e., 4n+2 delocalized electrons);

“halogen” or “halo” means fluoro (F), chloro (Cl), bromo (Br) or iodo (I); and

“Ar” means a monovalent cyclic radical having only carbon atoms and having aromatic character (i.e., 4n+2 delocalized electrons).

Thus, the term “cyclooxyalkyl” refers to, for example, tetrahydrofuran, tetrahydropyran, and oxepane.

The term “aromatic heterocyclic moiety” refers to, for example, oxadiazoles, pyridazines, pyrimidines, thiazoles, isothiazoles, oxazoles, isoxazoles, and furans.

The term “MCHR” refers to the melanin-concentrating hormone receptor protein 1 (MCHR1), unless otherwise stated.

The terms “treat”, “treating”, and “treatment” refer to modulation of a disease and/or its attendant symptoms.

The terms “prevent”, “preventing”, and “prevention” refer to decreasing or eliminating a disease and/or its attendant symptoms.

The terms “modulate”, “modulates”, “modulating”, or “modulation”, as used herein, refer to, for example, the activation (e.g., agonist activity) or inhibition (e.g., antagonist activity) of at least one MCHR.

The term “pharmaceutically-acceptable”, as employed herein, indicates the subject matter being identified as “pharmaceutically acceptable” is suitable and physiologically acceptable for administration to a patient/subject. For example, the term “pharmaceutically acceptable salt(s)” denotes suitable and physiologically acceptable salt(s).

The phrase “in vivo-hydrolysable precursor”, as employed herein, denotes a compound that, upon administration to a subject, undergoes chemical conversion via metabolic and/or chemical processes in vivo to yield a compound and/or derivative of Formula (J), or a pharmaceutically-acceptable salt thereof.

The terms “prophylax” or “prophylaxis”, as used herein, refer to (i) preventing the development of a disease and/or condition; and/or (ii) protecting against worsening of a disease and/or condition in a situation where the disease and/or condition has developed.

As used herein, the term “MCHR-mediated condition or disease” refers to a condition or disease amenable to modulation by an MCHR active agent.

The term “therapeutically-effective amount” refers to that amount of a compound sufficient to modulate one or more of the symptoms of the condition or disease being treated.

The term “anxiety disorder” refers to an emotional and/or behavioral disturbance characterized by persistent and pervasive worry or restlessness, and tension or irritability for no clear reason. An anxiety disorder may be accompanied by tachycardia or dyspnea. Exemplary anxiety disorders include, but are not limited to, for example, anxiety, generalized anxiety disorder, panic attacks, panic disorder, and obsessive-compulsive disorder (OCD).

The term “mood disorder” refers to an emotional and/or behavioral disturbance characterized by persistent and pervasive bouts of euphoria and/or depression. Exemplary mood disorders include, but are not limited to, for example, depression and bipolar disorders. Anxiety is frequently associated with mood disorders, such as, for example, depression.

### EXAMPLES

The invention is further defined in the following Examples. It should be understood that the Examples are given by way of illustration only. From the above discussion and the Examples, one skilled in the art can ascertain the essential characteristics of the invention, and without departing from the spirit and scope thereof, can make various changes and modifications to adapt the invention to various uses and conditions. As a result, the invention is not limited by the illustrative examples set forth hereinbelow, but rather defined by the claims appended hereto.

In general, the compounds of Formula I can be prepared in accordance with the general knowledge of one skilled in the art and/or using methods set forth in the Example and/or Intermediate sections that follow. Solvents, temperatures, pressures, and other reaction conditions can readily be selected by one of ordinary skill in the art. Starting materials are commercially available and/or readily prepared by one skilled in the art. Combinatorial techniques can be employed in the preparation of compounds, for example, where the intermediates possess groups suitable for these techniques.

It is of import to note that the names of the compounds disclosed herein were generated using AutoNom 2000 within ISIS/Draw. AutoNom (Automatic Nomenclature) is a chemical-name-generating program that assigns systematic IUPAC (International Union of Pure and Applied Chemistry) chemical names to drawn structures at the press of a button.

The following abbreviations are employed herein:

AcOH=Acetic acid

DMF=N,N-Dimethylformamide

DCM=Dichloromethane

DIEA=Diisopropyl ethyl amine

DMSO=Dimethylsulfoxide

EDC=N-(3-Dimethylaminopropyl)-N′-ethylcarbodiimide

EDCI=1-(3-Dimethylaminopropyl-)-3-ethylcarbodiimide hydrochloride

MeOH=Methanol

NMP=N-methylpyrrolidine

PS-CO₃²⁻=Polystyrene bound carbonate

PS-DIEA=Polystyrene bound diisopropyl ethyl amine

PS-CNBH4=Polystyrene bound cyano borohydride

rt=Room temperature

SiO₂=Silica gel

THF=Tetrahydrofuran

SFC=Supercritical fluid chromatography

### Intermediates

### Synthesis of Common Intermediates:

### Intermediate A

### 3-((1R,3R,5S)-8-Methyl-8-aza-bicyclo[3.2.1]oct-3-yloxy)-benzonitrile hydrochloride

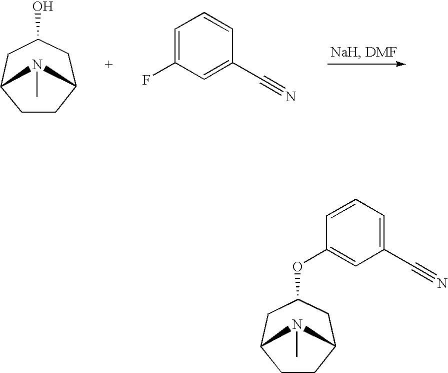

To a stirred solution of tropine hydrate (0.582 g, 4.1 mmol) in DMF (5 mL) was added NaH (0.2 g, 6.15 mmol of 60% mineral oil suspension) and the mixture stirred for 10 min. To this was added 3-fluorobenzenenitrile (0.50 g, 4.1 mmol) and the resultant slurry heated to 100° C. for 1 h. The material was then partitioned between ethyl acetate (70 mL) and H₂O (100 mL), and the organic layer was collected. The ethyl acetate layer was washed with brine (1×50 mL) and dried over Na₂SO₄. The material was filtered and concentrated to give Intermediate A as a colorless oil. The oil was dissolved in Et₂O and treated with 1 N HCl/Et₂O to afford the hydrochloride salt after filtration (0.40 g, 35%). ¹H NMR (300 MHz, DMSO-d₆) δ 1.91-1.96 (m, 2H), 2.23 (br s, 4H), 2.42-2.47 (m, 2H), 2.70 (s, 3H), 3.87 (br s, 2H), 4.74-4.80 (br s, 1H), 7.33 (dd, 1H, J=1.8 Hz, 7.5 Hz), 7.41 (d, 1H, J=7.5 Hz), 7.51-7.60 (m, 2H).

### Intermediate B

### 3-(1-Methyl-piperidin-4-yloxy)-benzonitrile hydrochloride

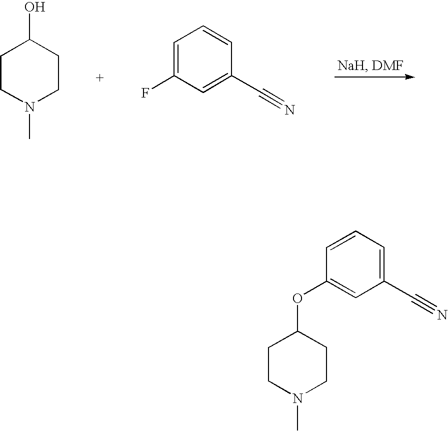

Intermediate B was prepared according to the method described for Intermediate A to give 0.900 g (27% yield) of Intermediate B as a white solid. ¹H NMR (300 MHz, DMSO-d₆) δ 1.58-1.68 (m, 2H), 1.89-1.98 (m, 2H), 2.18-2.21 (m, 2H), 2.59-2.62 (m, 2H), 3.28 (s, 3H), 4.44-4.51 (m, 1H), 7.29 (dd, 1H, J=1.8, 8.1 Hz), 7.37 (d, 1H, J=8.1 Hz), 7.43-7.49 (m, 2H). The material can be treated with PS-CO₃²⁻ (3 equiv) in CH₂Cl₂ for 3 h, filtered and concentrated to give the free base.

### Intermediate C

### 3-((1R,3R,5S)-8-Methyl-8-aza-bicyclo[3.2.1]oct-3-yloxy)-benzylamine dihydrochloride

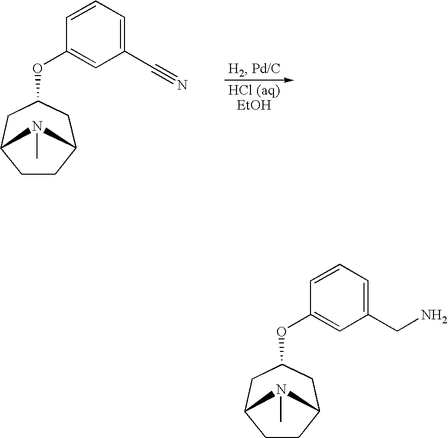

A solution of Intermediate A (400 mg) was dissolved in EtOH and treated with 10% Pd/C (˜200 mg), followed by conc. aq. HCl (0.1 mL). The material was shaken at 40 psi of H₂ pressure for 6 h, filtered and concentrated to dryness. The resulting Intermediate C residue was washed with diethyl ether and used as is without further purification. ¹H NMR (300 MHz, DMSO-d₆) δ 2.07-2.12 (m, 2H), 2.38 (br s, 4H), 2.55-2.59 (m, 2H), 2.68 (s, 3H), 3.86 (br s, 2H), 3.99 (br s, 2H), 4.70 (br s, 1H, 6.95 (dd, 1H, J=1.8 Hz, 7.8 Hz), 7.07 (d, 1H, J=7.2 Hz), 7.16 (br s, 1H), 7.34 (t, 1H, J=7.8 Hz), 8.44 (br s, 2H).

### Intermediate D

### 3-(1-Methyl-piperidin-4-yloxy)-benzylamine hydrochloride

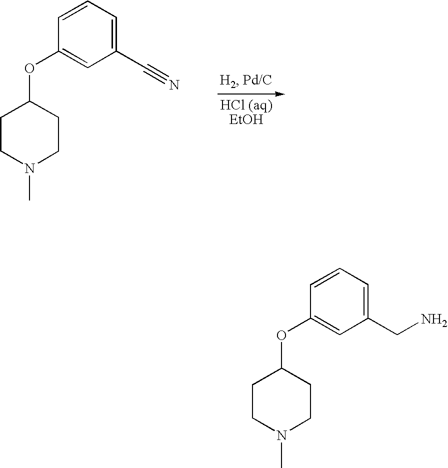

Prepared according to the method described for Intermediate C to give 0.032 g Intermediate D (15% yield). ¹H NMR (300 MHz, DMSO-d₆) δ 1.56-1.67 (m, 2H); 1.86-1.95 (m, 2H); 2.15-2.19 (m, 5H); 2.51-2.63 (m, 2H); 3.66 (s, 2H); 4.28-4.37 (m, 1H); 6.75 (dd, 1H, J=2.1, 8.1 Hz); 6.84 (d, 1H, J=7.5 Hz); 6.91 (s, 1H); 7.17 (t, 1H, J=7.8 Hz).

### Intermediate E

### Methyl-[3-(1-methyl-piperidin-4-yloxy)-benzyl]-amine

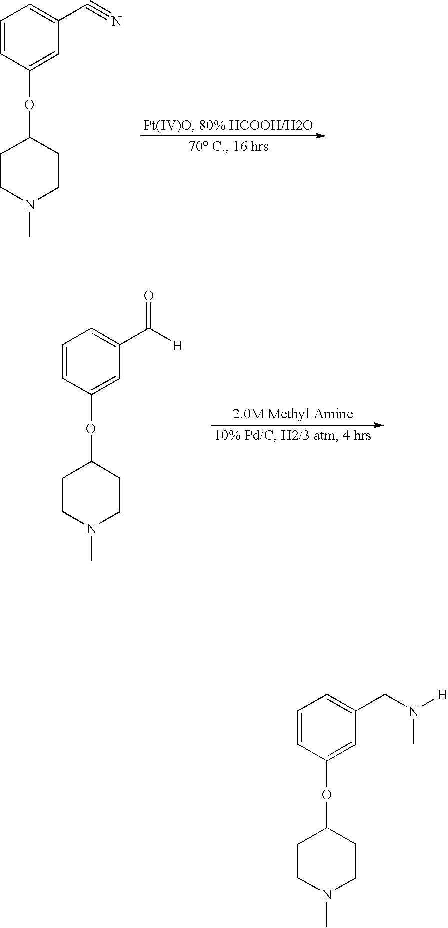

3-(1-Methyl-piperidin-4-yloxy)-benzonitrile (5 g, 23.1 mmol) was dissolved in an 80% solution of formic acid/H₂O. Pt(IV)O (0.524 g, 2.31 mmol) was added and the reaction mixture was allowed to stir and heat at 70° C. for 16 h. Next, the reaction was filtered and fresh Pt(IV)O (0.262 g, 1.15 mmol) was added. The reaction was allowed to continue stirring and heating for an additional 4 hours. LC/MS monitoring of the reaction indicated reaction completion at this point. The reaction mixture was filtered and the formic acid solution removed under vacuum. The residual light-yellow semi-solid was dissolved in methylene chloride and washed with saturated NaHCO₃, H₂O, and brine. The organic layer was dried over MgSO₄ and concentrated to yield 3.95 gm of the corresponding aldehyde 3-(1-methyl-piperidin-4-yloxy)-benzaldehyde, (77%). LC/MS [M+H]⁺ calculated: 220.29. found: 220.2. ¹H NMR (300 MHz, CDCl₃) δ 9.98 (s, 1H), 7.51 (m, 1H), 7.41 (d, 1H), 7.20 (m, 2H), 4.76 (m, 1H), 3.30 (m, 4H), 2.81 (s, 3H), 2.62 (m, 2H), 2.23 (m, 2H). Product determined to be clean enough to use without further purification. The aldehyde was dissolved in 50 mL of 2.0M methyl amine in MeOH. A catalytic amount of 10% Pd/C was added and mixture hydrogenated for 4 h at 3 atm. LC/MS monitoring of reaction indicated reaction completion. Reaction mixture was filtered and concentrated. Purification via SiO₂ chromatography using (9/0.9/0.1) mixture of (CH₂Cl₂/CH₃OH/NH₃OH) afforded 4.0 g of Intermediate E (90% yield). ¹H NMR (300 MHz, CDCl₃, 300K) δ 7.25 (t, 1H), 7.06 (s, 1H), 6.98 (d, 1H), 6.86 (d, 1H), 5.06 (s, 1H), 4.40 (m, 1H), 3.87 (d, 2H), 2.82 (m, 2H), 2.49 (m, 5H), 2.40 (s, 3H), 2.14 (m, 2H), 1.91 (m, 2H).

### Intermediate F

### Methyl-[3-((1R,3R,5S)-8-methyl-8-aza-bicyclo[3.2.1]oct-3-yloxy)-benzyl]-amine

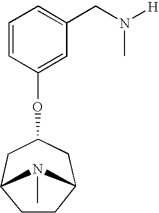

Prepared according to the method described for Intermediate E. ¹H NMR (300 MHz, CDCl₃) δ 7.21 (t, 1H), 6.83 (d, 1H), 6.81 (s, 1H), 6.86 (d, 1H), 6.71 (d, 1H), 4.53 (m, 1H), 3.71 (s, 2H), 3.11 (bs, 1H), 2.46 (s, 3H), 2.30 (s, 3H), 2.07 (m, 11H).

### Example 1

### 6-(4-Methoxy-phenyl)-N-[3-((1R,3R,5S)-8-methyl-8-aza-bicyclo[3.2.1]oct-3-yloxy)-benzyl]-nicotinamide

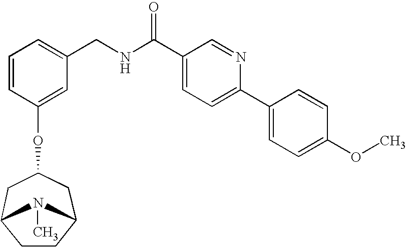

### 1A. 6-(4-Methoxy-phenyl)-nicotinic acid

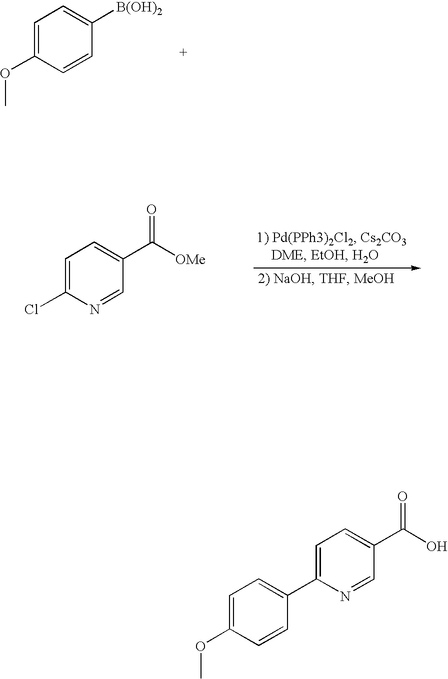

To a stirred solution of 6-chloro-nicotinic acid methyl ester (1 mmol, 0.216 g, 1 equiv.), in DME/EtOH/H₂O (7/2/1 mL) was added Cs₂CO₃ (2 mmol, 0.650 g, 2 equiv.) followed by 4-methoxyphenylboronic acid (1 mmol, 0.151 g, 1 equiv.), Pd(PPh₃)₂Cl₂ (approximately 10 mg) and the reaction stirred and heated to 90° C. for 1 hour. The reaction was concentrated and purified by chromatography (SiO2, gradient 100% CH₂Cl₂ to 98/2 CH₂Cl₂/MeOH). The residual material was hydrolyzed at 50° C. with 1.5 equivalent 1N NaOH, followed by neutralization with 1.5 equivalent 1 N HCl. The white solid that resulted was filtered and collected and then dried in vacuo at 65° C. overnight to give 0.120 g 1A. (50% yield).

### 1B. 6-(4-Methoxy-phenyl)-N-[3-((1R,3R,5S)-8-methyl-8-aza-bicyclo[3.2.1]oct-3-yloxy)-benzyl]-nicotinamide

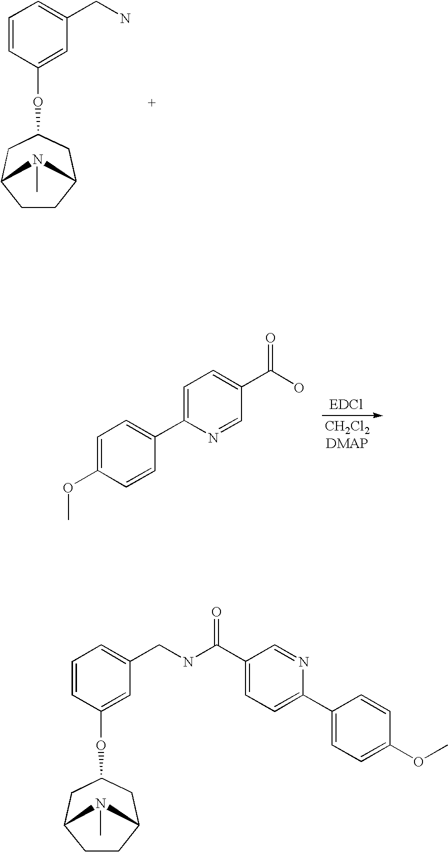

To a stirred solution of 1A (0.87 mmol, 0.200 g, 1.0 equiv.) in CH₂Cl₂ (10 mL) was added EDCI (1.0 mmol, 0.199 g, 1.20 equiv.) and the reaction stirred an additional 5 minutes. To this was added Intermediate C (0.87 mmol, 0.214 g, 1.0 equiv.) and DMAP (approximately 0.010 g). The reaction was stirred for 1 hour, diluted with CH₂Cl₂ (50 mL) and partitioned between water (10 mL). The organic layer was collected, dried (Na₂SO₄) and then chromatographed (SiO₂, 95/5 CH₂Cl₂/(2N NH₃ in MeOH) to give 0.230 g of 1B as a white solid (58% yield). ¹H NMR (300 MHz, DMSO-d₆) δ 1.79 (m, 2H), 1.91 (s, 4H), 1.97 (m, 2H), 2.16 (s, 3H), 3.00 (br s, 2H), 3.83 (s, 3H), 4.45 (d, 2H, J=5.7 Hz), 4.55 (m, 1H), 6.72 (d, 1H, J=8.0 Hz), 8.83 (s, 1H), 6.85 (d, 1H, J=8.0 Hz), 7.06 (d, 2H, J=8.7 Hz), 7.22 (t, 1H, J=8.0 Hz), 8.02 (d, 1H, J=8.4 Hz), 8.10 (d, 2H, J=8.7 Hz), 8.34 (d, 1H, J=8.4 Hz), 9.07 (d, 1H, J=1.8 Hz), 9.12 (m, 1H).

### Example 2

### N-[3-(1-Methyl-piperidin-4-yloxy)-benzyl]-6-phenyl-nicotinamide

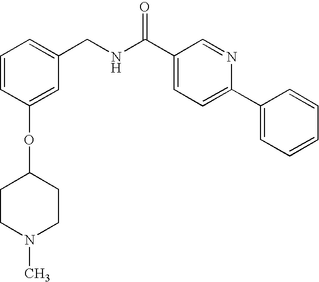

Prepared according to the method described for Example 1. ¹H NMR (300 MHz, CDCl₃) δ 9.05 (d, 1H), 8.19 (d, 1H), 8.03 (d, 2H), 7.79 (d, 1H), 7.46 (m, 3H), 7.25 (m, 1H), 6.92 (m, 2H), 6.85 (d, 1H), 6.81 (m, 1H), 2.28 (s, 3H), 2.03 (m, 10H).

### Example 3

### 4-(5-{[3-((1R,3R,5S)-8-Methyl-8-aza-bicyclo[3.2.1]oct-3-yloxy)-berzylamino]-methyl}-pyridin-2-yl)-benzonitrile

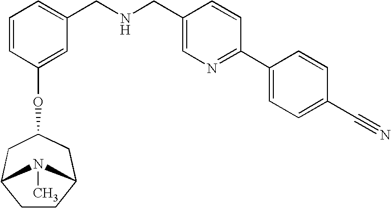

### 3A. (6-Bromo-pyridin-3-ylmethyl)-[3-((1R,3R,5S)-8-methyl-8-aza-bicyclo[3.2.1]oct-3-yloxy)-benzyl]-amine

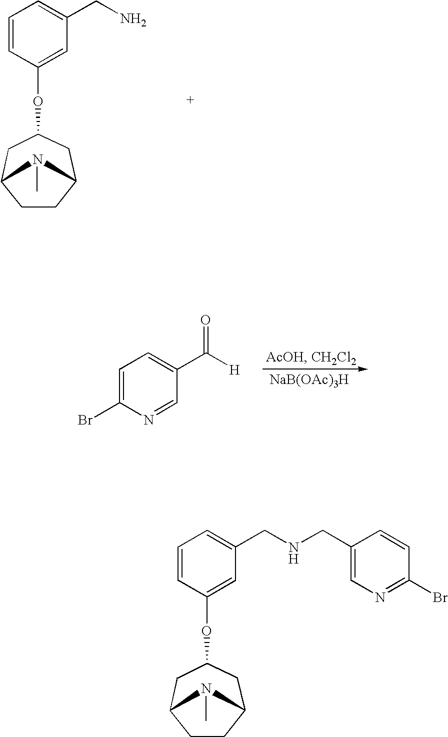

To a stirred solution of Intermediate C (5.0 mmol, 1.02 g, 1.25 equiv.) in CH₂Cl₂ was added 6-bromo-pyridine-3-carbaldehyde (3.3 mol, 0.62 g, 1.0 equiv.) and AcOH (6.6 mmol, 0.40 mL, 2.0 equiv.). The reaction was stirred for 20 min and then NaB(OAc)₃H (9.9 mmol, 1.05 g) was added. The reaction was stirred at RT for 4 h, diluted with CH₂Cl₂ (100 mL) and then partitioned between NaHCO₃. The organic residue was collected and the volatiles removed to give ˜1.0 g of 3A as a yellow foam. The material was used in the next reaction without further purification.

### 3B. 4-(5-{[3-((1R,3R,5S)-8-Methyl-8-aza-bicyclo[3.2.1]oct-3-yloxy)-benzylamino]-methyl}-pyridin-2-yl)-benzonitrile

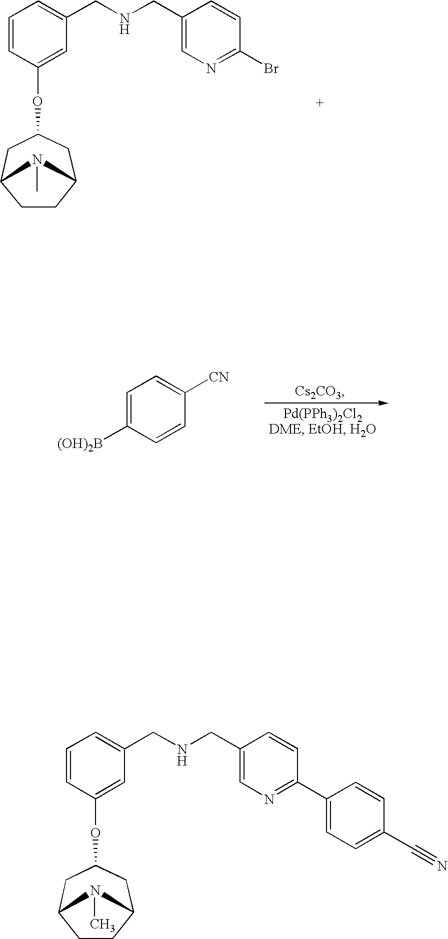

To a solution of 3A (0.24 mmol, 0.100 g, 1 equiv.), in DME/EtOH/H₂O (7/2/1 mL) was added Cs₂CO₃ (0.48 mmol, 0.156 g, 2 equiv.) followed by 4-cyanophenylboronic acid (0.48 mmol, 0.070 g, 2 equiv.), and Pd(PPh₃)₂Cl₂ (approximately 10 mg) and the reaction heated under microwave conditions (140° C. for 15 min). The reaction was concentrated and purified by preparatory HPLC chromatography (CH₃CN/H₂O gradient) to give 0.70 g of 3B as a colorless oil (70% yield). ¹H NMR (300 MHz, DMSO-d₆) δ 1.72 (m, 2H), 1.92 (s, 4H), 1.98 (m, 2H), 2.16 (s, 3H), 2.99 (m, 2H), 3.67 (s, 2H), 3.75 (s, 2H), 4.51 (m, 1H), 6.68 (d, 1H, J=8.1 Hz), 6.87 (m, 2H), 7.19 (t, 1H, J=7.5 Hz), 7.87-7.96 (m, 2H), 8.05 (d, 1H, J=8.4 Hz), 8.27 (d, 1H, J=8.4 Hz), 8.64 (s, 1H).

### Example 4

### [6-(4-Methoxy-phenyl)-pyridin-3-ylmethyl]-[3-((1R,3R,5S)-8-methyl-8-aza-bicyclo[3.2.1]oct-3-yloxy)-benzyl]-amine

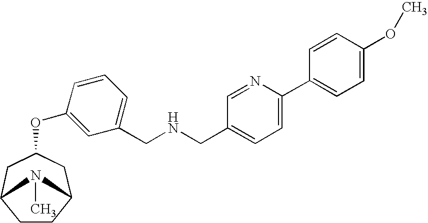

Prepared according to the method described for Example 3. ¹H NMR (300 MHz, DMSO-d₆) δ 2.08-2.13 (m, 2H), 2.22 (s, 4H), 2.43 (m, 2H), 2.70 (s, 3H), 3.85 (m, 5H), 4.21 (s, 2H), 4.33 (m, 2H), 4.71 (m, 1H), 7.00 (d, 1H, J=9.0 Hz), 7.10-7.14 (m, 3H), 7.20 (s, 1H), 7.37 (t, 1H, J=8.1 Hz), 8.09 (d, 2H, J=8.7 Hz), 8.25 (m, 1H), 8.30 (m, 1H), 8.82 (s, 1H).

### Example 5

### [3-((1R,3R,5S)-8-Methyl-8-aza-bicyclo[3.2.1]oct-3-yloxy)-benzyl]-[6-(4-trifluoromethoxy-phenyl)-pyridin-3-ylmethyl]-amine

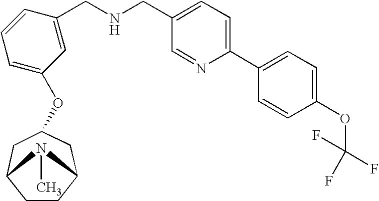

Prepared according to the method described for Example 3. ¹H NMR (300 MHz, DMSO-d₆) δ 1.88 (m, 2H), 2.02 (s, 4H), 2.10 (m, 2H), 2.35 (s, 3H), 3.05-3.08 (m, 2H), 3.68 (s, 2H), 3.74 (s, 2H), 4.65 (m, 1H), 6.70 (d, 1H, J=8.1 Hz), 6.89-6.91 (m, 2H), 7.22 (t, 1H, J=8.1 Hz), 7.45 (d, 2H, J=8.1 Hz), 7.86 (dd, 1H, J=7.8 Hz), 7.95 (d, 1H, J=8.1 Hz), 8.20 (d, 2H, J=8.7 Hz), 8.60 (s, 1H).

### Example 6

### [3-((1R,3R,5S)-8-Methyl-8-aza-bicyclo[3.2.1]oct-3-yloxy)-benzyl]-(6-phenyl-pyridin-3-ylmethyl)-amine

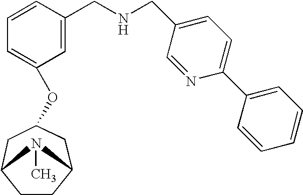

Prepared according to the method described for Example 3. ¹H NMR (300 MHz, DMSO-d₆) δ 1.84-1.86 (m, 2H), 2.0 (s, 4H), 2.07-2.12 (m, 2H), 2.30 (s, 3H), 3.17-3.25 (m, 2H), 3.68 (s, 2H), 3.74 (s, 2H), 4.58 (m, 1H), 6.73 (d, 1H, J=6.9 Hz), 6.84-6.91 (m, 2H), 7.22 (t, 1H, J=7.8 Hz), 7.41-7.50 (m, 3H), 7.81 (dd, 1H, J=8.1 Hz), 7.90 (d, 1H, J=8.1 Hz), 8.08 (d, 2H, J=8.4 Hz), 8.58 (s, 1H).

### Example 7

### [6-(4-Fluoro-phenyl)-pyridin-3-ylmethyl]-[3-((1R,3R,5S)-8-methyl-8-aza-bicyclo[3.2.1]oct-3-yloxy)-benzyl]-amine

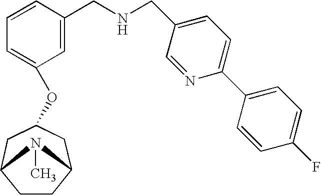

Prepared according to the method described for Example 3. ¹H NMR (300 MHz, DMSO-d₆) δ 1.88-1.92 (m, 2H), 2.00 (s, 4H), 2.10-2.16 (m, 2H), 2.41 (s, 3H), 3.28-3.30 (m, 2H), 3.68 (s, 2H), 3.73 (s, 2H), 4.60 (m, 1H), 6.75 (d, 1H, J=7.5 Hz), 6.90-6.92 (m, 2H), 7.22 (t, 1H, J=8.1 Hz), 7.27-7.33 (m, 2H), 7.83 (dd, J=8.1 Hz, 1.5 Hz), 8.10-8.14 (m, 1H), 8.58 (d, 1H, J=1.5 Hz).

### Example 8

### N-(3-(1-Methylpiperin-4-yloxy)benzyl)-5-(4-chlorophenyl)-N-methylisoxazole-3-carboxamide

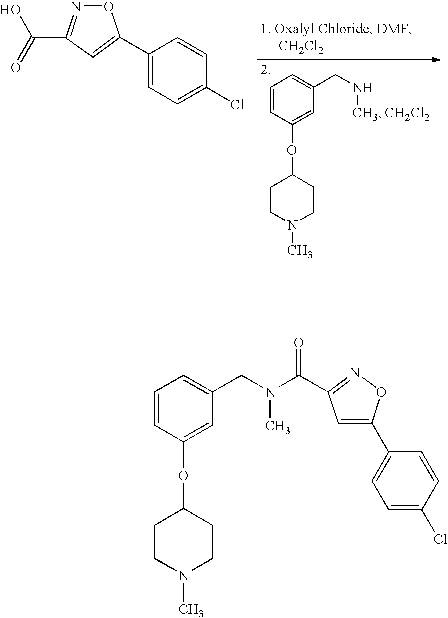

5-(4-Chlorophenyl)isoxazole-3-carboxylic acid (0.120 g, 0.5 mmol) was allowed to stir in 15 mL of CH₂Cl₂. Oxalyl chloride (0.192 ml, 1.5 mmol) was added and the reaction was allowed to stir. Two drops of DMF were added via pipet and the effervescing reaction mixture was allowed to stir two additional hours. The reaction mixture was concentrated to dryness and residual oxalyl chloride was azeotroped using CH₂Cl₂ (3×20 ml). The resulting light-yellow solid was allowed to dry under vacuum for 4 hours. The solid was dissolved in 10 ml of CH₂Cl₂, Intermediate E (0.100 g, 0.5 mmol) was added, and the reaction allowed to stir for 16 hours. LC/MS monitoring indicated reaction completion. The reaction was diluted with 20 mL of CH₂Cl₂, washed with brine (3×20 mL) and subsequently dried over MgSO₄. Concentration to dryness yielded an off-white solid. Purification via SiO₂ chromatography using a (9/0.9/0.1) mixture of (CH₂Cl₂/CH₃OH/NH₃OH) afforded 34.2 mgs (20% yield) of the title compound as a white solid. ¹H NMR (300 MHz, CDCl₃) δ 7.72 (m, 2H), 7.45 (m, 2H), 7.25 (m, 1H), 6.85 (m, 4H), 4.80 (d, 2H), 4.30 (m, 1H), 3.10 (d, 3H), 2.68 (m, 2H), 2.30 (m, 5H), 1.98 (m, 2H), 1.83 (m, 2H).

### Example 9

### 5-(4-Methoxy-phenyl)-isoxazole-3-carboxylic acid 3-(1-methyl-piperidin-4-yloxy)-benzylamide

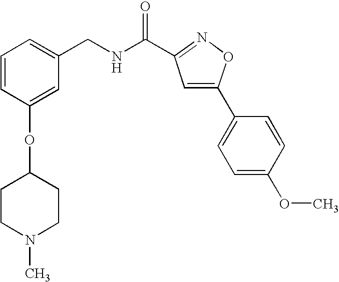

Prepared according to the method described for Example 8. ¹H NMR (300 MHz, CDCl₃) 1.75-1.90 (m, 2H), 1.9-2.1 (m, 2H), 2.30 (m, 3H), 2.6-2.75 (m, 2H), 3.87 (s, 3H), 4.25-4.37 (m, 1H), 4.60 (d, 2H, J=5.7 Hz), 6.86-6.91 (m, 5H), 6.98-7.01 (m, 2H), 7.74-7.77 (m, 2H).

### Example 10

### 5-(4-Methoxy-phenyl)-furan-2-carboxylic acid 3-(1-methyl-piperidin-4-yloxy)-benzylamide

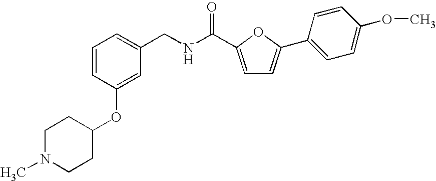

Prepared according to the method described for Example 8. ¹H NMR (300 MHz, CDCl₃, 300K) 1.81-1.89 (m, 2H), 1.96-2.03 (m, 2H), 2.24-2.30 (m, 5H), 2.65-2.68 (m, 2H), 3.84 (s, 3H), 4.30-4.34 (m, 1H), 4.62 (d, 2H, J=6.0), 6.60-6.61 (m, 2H), 6.83-6.86 (m, 1H), 6.92-6.96 (m, 4H), 7.20-7.21 (m, 1H), 7.60-7.63 (m, 2H).

### Example 11

### 5-(4-Methoxy-phenyl)-isoxazole-3-carboxylic acid 3-((1R,3R,5S)-8-methyl-8-aza-bicyclo[3.2.1]oct-3-yloxy)-benzylamide

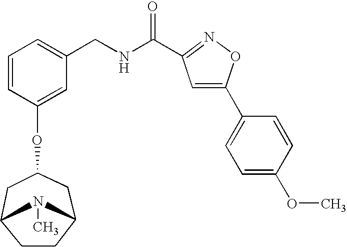

Prepared according to the method described for Example 8. ¹H NMR (300 MHz, CDCl₃) 1.96-2.09 (m, 9H), 2.30 (s, 1H,) 3.05-3.15 (m, 2H), 3.87 (s, 3H), 4.45-4.55 (m, 1H), 4.60 (d, 2H, J=6.0), 6.81-7.15 (m, 7H), 7.72-7.75 (m, 2H).

### Example 12

### 5-(4-Methoxy-phenyl)-furan-2-carboxylic acid 3-((1R,3R,5S)-8-methyl-8-aza-bicyclo[3.2.1]oct-3-yloxy)-benzylamide

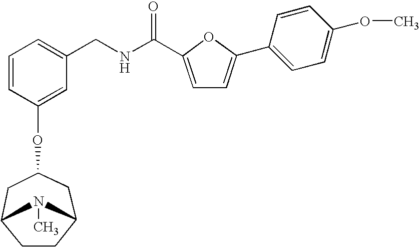

Prepared according to the method described for Example 8. ¹H NMR (300 MHz, CDCl₃) 1.91-2.14 (m, 9H), 2.29 (s, 3H), 3.11 (m, 2H), 3.84 (s, 3H), 4.52 (t, 1H,), 4.61 (d, 2H, J=6.0) 6.60-6.61 (m, 2H), 6.74-6.84 (m, 2H), 6.91-6.94 (m, 3H), 7.20 (m, 1H), 7.59-7.62 (m, 2H).

### Example 13

### 5-(4-Chloro-phenyl)-isoxazole-3-carboxylic acid 3-(1-methyl-piperidin-4-yloxy)-benzylamide

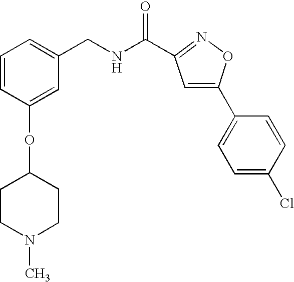

Prepared according to the method for Example 8. ¹H NMR (300 MHz, CDCl₃) δ 7.74 (d, 2H), 7.48 (d, 2H), 7.26 (m, 2H), 6.98 (m, 1H), 6.85 (m, 3H), 4.62 (d, 2H), 4.32 (m, 1H), 2.68 (m, 2H), 2.30 (m, 5H), 1.97 (m, 2H), 1.88 (m, 2H).

### Example 14

### 5-(4-Chloro-phenyl)-isoxazole-3-carboxylic acid methyl-[3-((1R,3R,5S)-8-methyl-8-aza-bicyclo[3.2.1]oct-3-yloxy)-benzyl]-amide

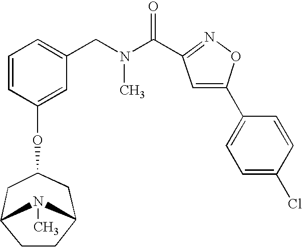

Prepared according to the method described for Example 8. ¹H NMR (300 MHz, CDCl₃) δ 7.72 (d, 2H), 7.48 (m, 2H), 7.27 (m, 2H), 6.80 (m, 1H), 4.88 (d, 2H), 4.51 (m, 1H), 3.25 (d, 5H), 2.29 (d, 3H), 2.03 (m, 10H).

### Example 15

### 5-(4-Chloro-phenyl)-isoxazole-3-carboxylic acid 3-((1R,3R,5S)-8-methyl-8-aza-bicyclo[3.2.1]oct-3-yloxy)-benzylamide

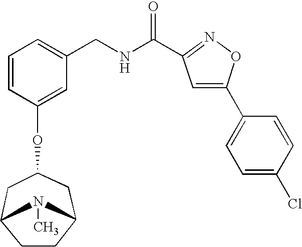

Prepared according to the method described for Example 8. ¹H NMR (300 MHz, DMSO-d6) δ 7.98 (d, 2H), 7.65 (d, 2H), 7.44 (s, 1H), 7.27 (t, 1H), 6.90 (m, 3H), 4.67 (bs, 1H), 4.45 (d, 2H), 3.83 (bs, 1H), 2.66 (s, 3H), 2.22 (m, 10H).

### Example 16

### N-[3-((1R,3R,5S)-8-Methyl-8-aza-bicyclo[3.2.1]oct-3-yloxy)-benzyl]-6-phenoxy-nicotinamide

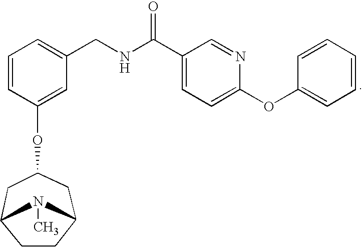

Prepared according to the method described for Example 8. ¹H NMR (300 MHz, CDCl₃) δ 8.61 (d, 1H), 8.15 (m, 1H), 7.39 (t, 2H), 7.21 (t, 1H), 7.13 (d, 1H), 6.80 (m, 5H), 4.57 (d, 2H), 4.50 (m, 1H), 3.16 (bs, 1H), 2.32 (s, 2H), 2.07 (m, 10H).

### Example 17

### 5-Phenyl-2H-pyrazole-3-carboxylic acid 3-((1R,3R,5S)-8-methyl-8-aza-bicyclo[3.2.1]oct-3-yloxy)-benzylamide

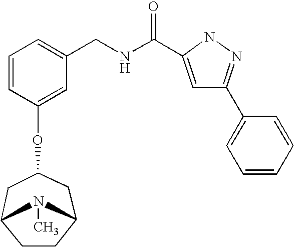

Prepared according to the method described for Example 8. ¹H NMR (300 MHz, CDCl₃) δ 7.65 (d, 2H), 7.38 (m, 4H), 7.18 (t, 1H), 7.08 (s, 1H), 6.91 (d, 1H), 6.78 (bs, 1H), 6.69 (d, 1H), 4.59 (d, 2H), 4.44 (m, 1H), 3.47 (s, 1H), 3.16 (bs, 1H), 2.30 (s, 3H), 2.10 (m, 10H).

### Example 18

### 5-Phenyl-2H-pyrazole-3-carboxylic acid 3-(1-methyl-piperidin-4-yloxy)-benzylamide

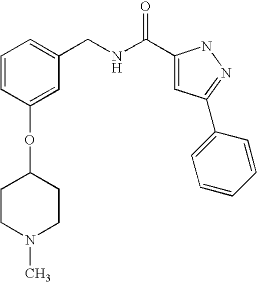

Prepared according to the method described for Example 8. ¹H NMR (300 MHz, CDCl₃) δ 7.61 (d, 2H), 7.36 (m, 4H), 7.19 (t, 1H), 7.08 (s, 1H), 6.89 (m, 1H), 6.80 (d, 1H), 4.60 (d, 2H), 4.27 (m, 1H), 2.67 (m, 2H), 2.27 (m, 5H), 1.94 (m, 2H), 1.91 (m, 2H).

### Example 19

### 6-Chloro-N-[3-((1R,3R,5S)-8-methyl-8-aza-bicyclo[3.2.1]oct-3-yloxy)-benzyl]-nicotinamide

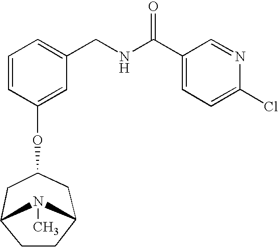

Prepared according to the method described for Example 8. ¹H NMR (300 MHz, CDCl₃) δ 8.73 (d, 1H), 8.10 (d, 1H), 7.40 (d, 1H), 7.24 (t, 1H), 6.89 (d, 1H), 6.79 (m, 2H), 4.59 (d, 2H), 4.49 (m, 1H), 3.09 (bs, 1H), 2.28 (s, 3H), 2.03 (m, 10H).

### Example 20

### [3-(1-Methyl-piperidin-4-yloxy)-benzyl]-(6-phenoxy-pyridin-3-ylmethyl)-amine

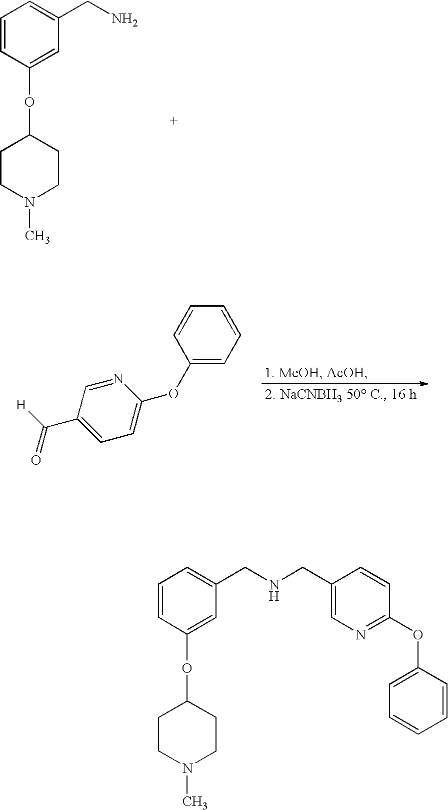

6-Phenoxynicotinaldehyde (0.054 g, 0.272 mmol) and Intermediate D (0.100 g, 0.406 mmol) were allowed to stir in 15 mL of MeOH. Glacial acetic acid was added via pipet until a pH of ˜4.5-5.0 was reached. The reaction was allowed to stir and heat at 50° C. for twenty min. Sodium cyanoborohydride (0.014 g, 0.2 mmol) was added and the reaction was allowed to continue stirring and heating for 16 h. LC/MS monitoring of reaction indicated reaction completion. The reaction was quenched with 1 mL H₂O and concentrated to dryness. The residue re-dissolved in methylene chloride and washed with Brine (3×15 mL). The organic layer was dried over MgSO₄ concentrated to dryness. Purification via SiO₂ chromatography using a (9/0.9/0.1) mixture of (CH₂Cl₂/CH₃OH/NH₃OH) afforded 42 mgs (23%) of the desired target compound. ¹H NMR (300 MHz, CDCl₃) δ 8.13 (d, 1H), 7.70 (m, 1H), 7.39 (m, 2H), 7.18 (m, 4H), 6.87 (m, 4H), 4.32 (m, 1H), 3.75 (d, 4H), 2.69 (m, 2H), 2.30 (m, 5H), 19.7 (m, 2H), 1.85 (m, 2H), 1.70 (bs, 2H).

### Example 21

### [5-(4-Fluoro-phenyl)-isoxazol-3-ylmethyl]-[3-((1R,3R,5S)-8-methyl-8-aza-bicyclo[3.2.1]oct-3-yloxy)-benzyl]-amine

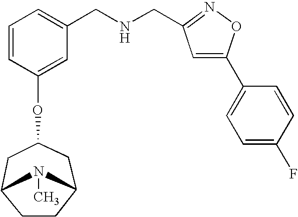

Prepared according to the method described for Example 20. ¹H NMR (300 MHz, CDCl₃) δ 7.78 (m, 2H), 7.20 (m, 3H), 6.91 (d, 1H), 6.83 (bs, 1H), 6.74 (d, 1H), 6.48 (s, 1H), 4.53 (m, 1H), 3.91 (d, 4H), 3.11 (bs, 1H), 2.30 (s, 3H), 2.03 (m, 10H).

### Example 22

### [3-((1R,3R,5S)-8-Methyl-8-aza-bicyclo[3.2.1]oct-3-yloxy)-benzyl]-(6-phenoxy-pyridin-3-ylmethyl)-amine

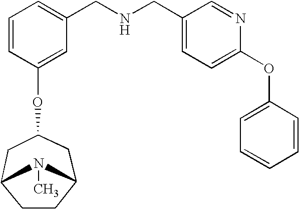

Prepared according to the method described for Example 20. ¹H NMR (300 MHz, CDCl₃) δ 8.13 (s, 1H), 7.72 (m, 1H), 7.41 (t, 2H), 7.18 (m, 4H), 6.88 (d, 2H), 6.81 (bs, 1H), 6.72 (d, 1H), 4.53 (m, 1H), 3.76 (d, 4H), 3.14 (bs, 1H), 2.32 (s, 3H), 2.05 (m, 10H).

### Example 23

### [5-(4-Chloro-phenyl)-isoxazol-3-ylmethyl]-methyl-[3-((1R,3R,5S)-8-methyl-8-aza-bicyclo[3.2.1]oct-3-yloxy)-benzyl]-amine

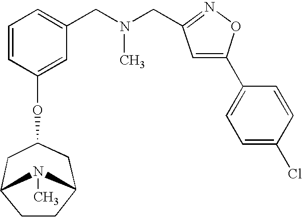

Prepared according to the method described for Example 20. ¹H NMR (300 MHz, CDCl₃) δ 7.71 (d, 2H), 7.44 (d, 2H), 7.22 (m, 1H), 6.92 (d, 1H), 6.85 (bs, 1H), 6.74 (d, 1H), 6.54 (s, 1H), 4.53 (m, 1H), 3.64 (d, 4H), 3.10 (bs, 1H), 2.30 (d, 6H), 2.01 (m, 10H).

### Example 24

### 5-(3-Methoxyphenyl)pyridine-2-carboxylic acid 3-((1R,3R,5S)-8-methyl-8-aza-bicyclo[3.2.1]oct-3-yloxy)benzylamide

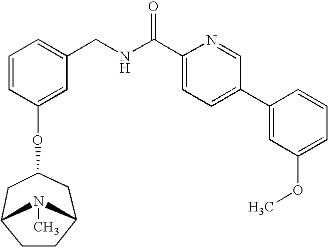

### 24A. 5-Bromopyridine-2-carboxylic acid 3-((1R,3R,5S)-2,3-dimethyl-2-aza-bicyclo[2.2.1]hept-5-yloxy)benzylamide

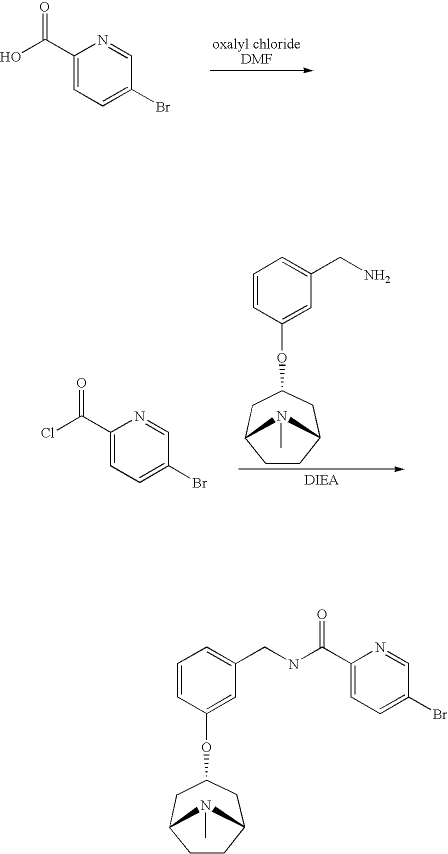

To a stirred solution/suspension of 5-bromo-2-pyridinecarboxylic acid (0.2 g, 1 mmol) in CH₂Cl₂ (25 mL) was added oxalyl chloride (0.26 mL, 3 mmol) and 1 drop of DMF. Gas evolved and the all the solids dissolved. After 1 h the reaction was evaporated and pumped. This material was dissolved/suspended in CH₃CN (10 mL) then Intermediate C (0.25 g, 1 mmol) and diisopropylethylamine (2 mL) were added. A white solid formed (DIEA.HCl). After 1.5 h the reaction was filtered and the filtrate was evaporated, taken up in CH₂Cl₂, filtered and chromatographed on silica gel, eluting with a gradient of 0-10% methanol+1% ammonium hydroxide in CH₂Cl₂. The product fractions were evaporated, dissolved in CH₂Cl₂, washed with 1 M NaOH (to remove residual hydrochloride salt) and evaporated to afford 0.25 g of 24A (58% yield). ¹H NMR (300 MHz, DMSO-d₆) δ 9.30 (t, J=6.4 Hz, 1H), 8.78 (d, J=2.1 Hz, 1H), 8.25 (dd, J=2.3 Hz, J=8.2 Hz, 1H), 7.98 (d, J=8.3 Hz, 1H), 7.19 (t, J=7.8 Hz, 1H), 6.88-6.80 (m, 2H), 6.74-6.68 (m, 1H), 4.50 (t, J=4.9 Hz, 1H), 4.43 (d, J=6.4 Hz, 2H), 3.03-2.96 (m, 2H), 2.52-2.47 (m, 3H), 2.03-1.87 (m, 6H), 1.77-1.68 (m, 2H).

### 24B. 5-(3-Methoxyphenyl)pyridine-2-carboxylic acid 3-((1R,3R,5S)-8-methyl-8-aza-bicyclo[3.2.1]oct-3-yloxy)benzylamide

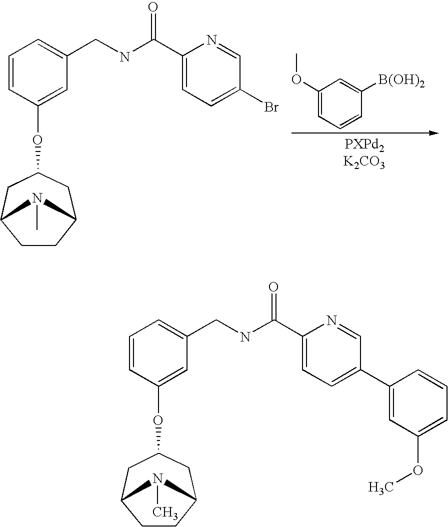

24A (0.11 g, 0.25 mmol) and 3-methoxyphenylboronic acid (0.063 g, 0.5 mmol) were combined in 4 mL DME/H₂O/EtOH (7/3/2) and K₂CO₃ (0.14 g, 1 mmol), and CombiPhos PXPd₂ catalyst (7 mg) was added. This mixture was heated via microwave at 150° C. for 5 nm in. The reaction separated into 2 phases, the organic (upper) phase was filtered and evaporated. This was dissolved/suspended in CH₂Cl₂ (4 mL) and filtered. The filtrate was chromatographed on SiO₂, eluting with a gradient of 0-10% MeOH+1% NH₄OH in CH₂Cl₂ to afford 0.108 g 24B (92% yield). ¹H NMR (300 MHz, DMSO-d₆) δ 9.29 (t, J=6.4 Hz, 1H), 8.95 (d, J=1.6 Hz, 1H), 8.29 (dd, J=8.1 Hz, J=2.3 Hz, 1H), 8.11 (d, J=8.2 Hz, 1H), 7.49-7.42 (m, 1H), 7.39-7.32 (m, 2H), 7.21 (t, J=7.8 Hz, 1H), 7.07-7.02 (m, 1H), 6.90-6.83 (m, 2H), 6.75-6.69 (m, 1H), 4.55-4.44 (m, 3H), 3.85 (s, 3H), 3.08-2.98 (m, 2H), 2.18 (s, 3H), 2.06-1.87 (m, 6H), 1.80-1.70 (m, 2H).

### Example 25

### N-(3-(1-Methylpiperidin-4-yloxy)benzyl)-5-(4-methoxyphenyl)pyridine-2-carboxamide

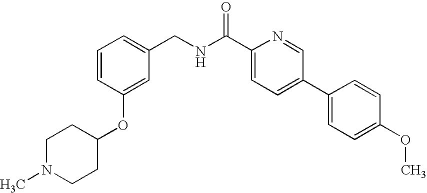

Prepared according to the method described for Example 24. ¹H NMR (300 MHz, DMSO-d₆) δ 9.27 (t, J=6.4 Hz, 1H), 8.91 (d, J=2.0 Hz, 1H), 8.23 (dd, J=8.2 Hz, J=2.3 Hz, 1H), 8.08 (d, J=8.1 Hz, 1H), 7.79-7.74 (m, 2H), 7.20 (t, J=7.8 Hz, 1H), 7.12-7.07 (m, 2H), 6.92-6.86 (m, 2H), 6.83-6.78 (m, 1H), 4.47 (d, J=6.4 Hz, 2H), 4.35-4.26 (m, 1H), 3.83 (s, 3H), 2.63-2.55 (m, 2H), 2.19-2.10 (m, 5H), 1.95-1.85 (m, 2H), 1.67-1.53 (m, 2H).

### Example 26

### N-(3-(1-Methylpiperidin-4-yloxy)benzyl)-5-phenylpyridine-2-carboxamide

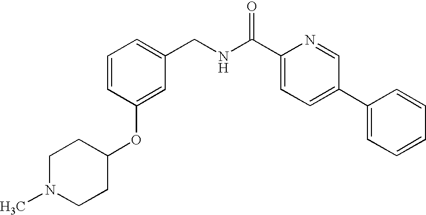

Prepared according to the method described for Example 24. ¹H NMR (300 MHz, DMSO-d₆) δ 9.30 (t, J=6.3 Hz, 1H), 8.96 (d, J=2.3 Hz, 1H), 8.28 (dd, J=8.2 Hz, J=2.3 Hz, 1H), 8.12 (d, J=8.1 Hz, 1H), 7.84-7.77 (m, 2H), 7.58-7.43 (m, 3H), 7.21 (t, J=7.8 Hz, 1H), 6.93-6.78 (m, 3H), 4.48 (d, J=6.4 Hz, 2H), 4.36-4.25 (m, 1H), 2.65-2.55 (m, 2H), 2.21-2.09 (m, 5H), 1.97-1.84 (m, 2H), 1.67-1.52 (m, 2H).

### Example 27

### 5-(4-Methoxyphenyl)pyridine-2-carboxylic acid 3-((1R,3R,5S)-8-methyl-8-aza-bicyclo[3.2.1]oct-3-yloxy)benzylamide

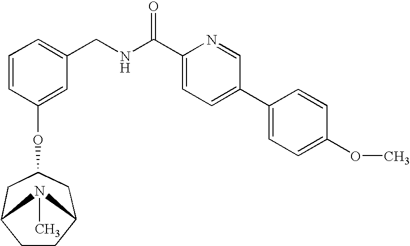

Prepared according to the method described for Example 24. ¹H NMR (300 MHz, DMSO-d₆) δ 9.25 (t, J=6.3 Hz, 1H), 8.91 (d, J=1.7 Hz, 1H), 8.23 (dd, J=8.5 Hz, J=2.3 Hz, 1H), 8.07 (d, J=8.4 Hz, 1H), 7.79-7.73 (m, 2H), 7.21 (t, J=7.8 Hz, 1H), 7.13-7.06 (m, 2H), 6.90-6.82 (m, 2H), 6.75-6.70 (m, 1H), 4.56-4.50 (m, 1H), 4.47 (d, J=6.9 Hz, 2H), 3.82 (s, 3H), 3.10-3.00 (m, 2H), 2.23-2.15 (m, 3H), 2.07-1.88 (m, 6H), 1.81-1.70 (m, 2H).

### Example 28

### 5-(3-Methoxyphenyl)pyridine-2-carboxylic acid 3-(1-methylpiperidin-4-yloxy)benzylamide

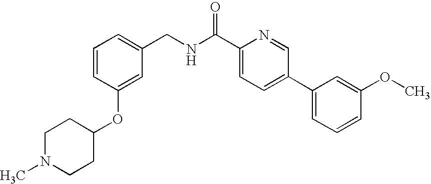

Prepared according to the method described for Example 24. ¹H NMR (300 MHz, DMSO-d₆) δ 9.29 (t, J=7.2 Hz, 1H), 8.95 (d, J=1.5 Hz, 1H), 8.30 (dd, J=8.1 Hz, J=2.4 Hz, 1H), 8.13-8.09 (m, 1H), 7.49-7.41 (m, 1H), 7.38-7.32 (m, 2H), 7.24-7.17 (m, 1H), 7.07-7.02 (m, 1H), 6.92-6.86 (m, 2H), 6.84-6.79 (m, 1H), 4.48 (d, J=6.4 Hz, 2H), 4.35-4.25 (m, 1H), 3.85 (s, 3H), 2.65-2.55 (m, 2H), 2.20-2.08 (m, 5H), 1.96-1.85 (m, 2H), 1.67-1.54 (m, 2H).

### Example 29

### 5-Phenylpyridine-2-carboxylic acid 3-((1R,3R,5S)-8-methyl-8-aza-bicyclo[3.2.1]oct-3-yloxy)benzylamide

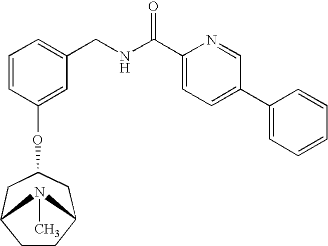

Prepared according to the method described for Example 24. ¹H NMR (300 MHz, DMSO-d₆) δ 9.29 (t, J=6.5 Hz, 1H), 8.95 (d, J=2.2 Hz, 1H), 8.25 (dd, J=9.2 Hz, J=2.1 Hz, 1H), 8.12 (d, J=8.5 Hz, 1H), 7.84-7.76 (m, 2H), 7.59-7.44 (m, 3H), 7.21 (t, J=7.7 Hz, 1H), 6.91-6.83 (m, 2H), 6.75-6.68 (m, 1H), 4.55-4.44 (m, 3H), 3.05-2.96 (m, 2H), 2.17 (s, 3H), 2.04-1.88 (m, 6H), 1.79-1.70 (m, 2H).

### Example 30

### 5-(4-Methoxy-phenyl)-[1,2,4]oxadiazole-3-carboxylic acid 3-((1R,3R,5S)-8-methyl-8-aza-bicyclo[3.2.1]oct-3-yloxy)-benzylamide

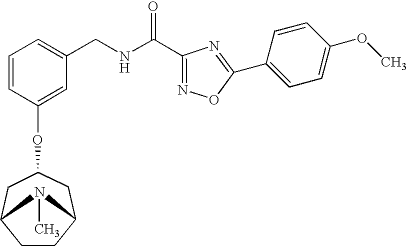

### 30A. 5-(4-Methoxy-phenyl)-[1,2,4]oxadiazole-3-carbonyl chloride

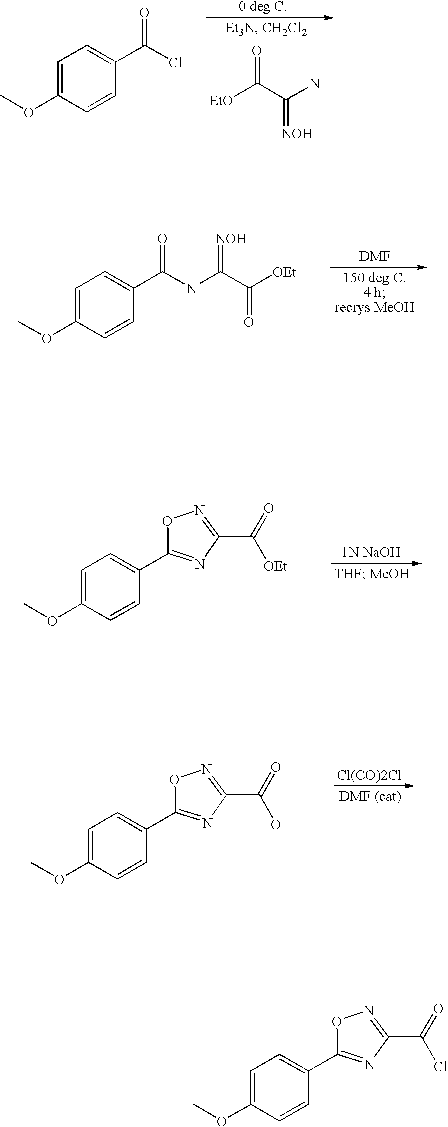

To a stirred solution of 4-methoxybenzoyl chloride (9.0 g, 53 mmol) in CH₂Cl₂ at 0° C., was slowly added a CH₂Cl₂ (10 mL) solution of ethyl 2-oximo-oxamate (7.0 g, 53 mmol) and Et₃N (7.4 mL, 53 mmol). The reaction was stirred for 1 h and allowed to warm to rt. The reaction was partitioned between NaHCO₃ (sat), and then NaCl (sat.). The organic layer was dried with Na₂SO₄, filtered and rotary evaporated to give a white solid (˜15 g). The white solid was then dissolved in DMF (100 mL) and heated to 150° C. for 4 h or until all was complete by LC-MS. The DMF was diluted with EtOAc (1 L) and extracted with H₂O (4×500 mL), and NaCl (sat.). The organic layer was dried (Na₂SO₄), filtered and rotary evaporated to give the crude ester. The ester was recrystallized from MeOH (˜100 mL) to give pure material (˜10.2 g still moist with MeOH). The material was then dissolved in THF (50 mL) and then MeOH (30 mL) and 1 N NaOH (53 mL) was added. After five minutes, the reaction was determined to be complete by LC-MS. The volatiles were then removed by rotary evaporation, and the residual aqueous suspension was quenched with 1 N HCl (53 mL). The resultant solid was filtered, and dried overnight at 60° C. under vacuum to give a white solid (6.7 g, 76%). The resultant solid was stirred in CH₂Cl₂, a catalytic drop of DMF added followed by oxalyl chloride (1.3 equiv.). The reaction is typically complete in 20-30 min and after removal of the solvents under high vacuum, 30A can be used in the next reaction without further purification.

### 30B. 5-(4-Methoxy-phenyl)-[1,2,4]oxadiazole-3-carboxylic acid 3-((1R,3R,5S)-8-methyl-8 aza-bicyclo[3.2.1]oct-3-yloxy)-benzylamide

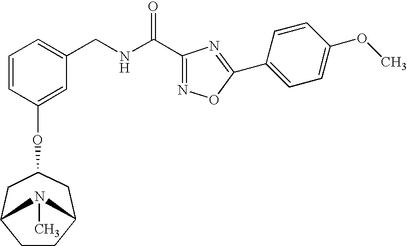

30B is produced by reacting 30A in a procedure similar to the procedure set forth in Example 8. ¹H NMR (CDCl₃) δ 1.92-2.15 (m, 8H), 2.30 (s, 3H), 3.12 (br s, 2H), 3.90 (s, 3H), 4.54-4.52 (m, 1H), 4.65 (d, J=6.0 Hz, 2H), 6.77 (d, J=7.5 Hz, 1H), 6.84 (s, 1H), 6.92 (d, J=7.5 Hz, 1H), 7.03 (d, J=11.5 Hz, 2H), 7.28-7.25 (m, 1H), 8.11 (d, J=11.5 Hz, 2H).

1. A compound in accord with Formula I: 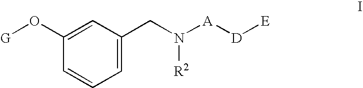
wherein:
G is: 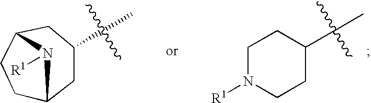
R¹ is H, —C₁₋₆alkyl, C₁₋₆haloalkyl, —C₃₋₈cycloalkyl, or —C₃₋₈cyclooxyalkyl;
R² is hydrogen or —C₁₋₄alkyl;
A is —CH₂— or —C(═O)—,
D is a 5- or 6-membered aromatic heterocyclic moiety having 1, 2 or 3 heteroatoms selected from nitrogen, oxygen or sulfur where not more than one of said heteroatoms is oxygen or sulfur, or an 8-, 9- or 10-membered fused aromatic heterocyclic moiety having 1, 2 or 3 heteroatoms selected from nitrogen, oxygen or sulfur where not more than one of said heteroatoms is oxygen or sulfur;
E is a hydrogen, halogen, —CN, —NO₂, —CF₃, —CONR⁷R⁸—S(O)ₙR⁷—NR⁷R⁸, —CH₂NR⁷R⁸, —OR⁷, —CH₂OR⁷—NC(═O)R⁷, —CO₂R⁷, —C₁₋₆alkyl —C₂₋₆alkenyl —C₂₋₆alkynyl, C₁₋₆alkoxy, C₃₋₈cycloalkyl —O—CH₂—O— or -J-Ar;
J is —O—, —CH₂—, —O—CH₂— or a bond;
Ar is a 5- or 6-membered aromatic or heteroaromatic moiety having 0, 1 or 2 nitrogen atoms, 0 or 1 oxygen atoms, and 0 or 1 sulfur atoms, or an 8-, 9- or 10-membered fused aromatic or heteroaromatic ring system having 0, 1, 2 or 3 nitrogen atoms, 0 or 1 oxygen atoms, and 0 or 1 sulfur atoms;
wherein Ar is unsubstituted or has 1, 2 or 3 substituents independently selected at each occurrence from —C₁₋₆alkyl, —C₂₋₆alkenyl, —C₂₋₆alkynyl, halogen, —CN, —NO₂, —CF₃, —CONR⁷R⁸, —S(O)ₙR⁷, —NR⁷R⁸, —CH₂NR⁷R⁸, —OR⁷, —CH₂OR⁷, —NC(═O)R⁷, and —CO₂R⁷;
n is 1, 2, or 3; and
R⁷ and R⁸ are each independently hydrogen, CF₃, C₁₋₆alkyl and/or C₃₋₈cycloalkyl;
or in vivo-hydrolysable precursors or pharmaceutically-acceptable salts thereof.

2-6. (canceled)

7. A compound according to claim 1, wherein A is —C(═O)—; or an in vivo-hydrolysable precursor or a pharmaceutically-acceptable salt thereof.

8. A compound according to claim 1, wherein A is —CH₂—; or an in vivo-hydrolysable precursor or a pharmaceutically-acceptable salt thereof.

9. A compound according to claim 1, wherein:
R¹ is selected from H or C₁₋₄alkyl;
D is selected from a moiety of formula II III, IV, V, VI, or VII: 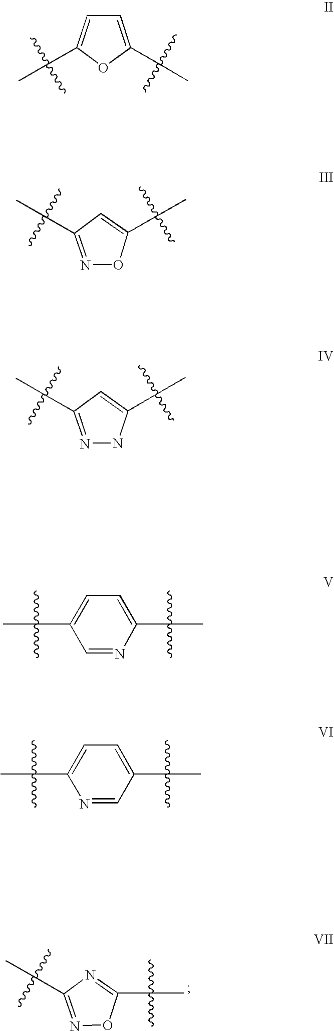
E is -J-Ar or halogen;
J is —O— or a bond; and
Ar is as heretofore defined;
or an in vivo-hydrolysable precursor or a pharmaceutically-acceptable salt thereof.

10. A compound according to claim 1, wherein:
G is: 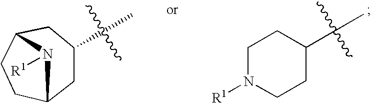
R¹ is H or methyl;
R² is hydrogen or methyl;
A is —C(═O)—;
D is a moiety of formula II, IV, V, VI, or VII: 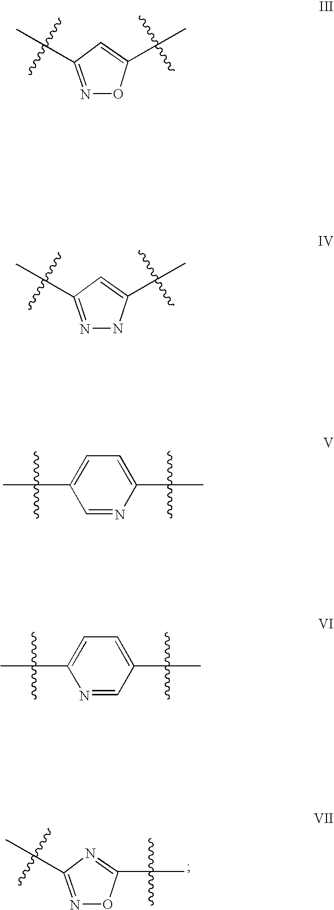
E is J-Ar;
J is a bond; and
Ar is a phenyl, phenoxy or phenyl substituted with —Cl or —O—CH₃;
or an in vivo-hydrolysable precursor or a pharmaceutically-acceptable salt thereof.

11. A compound according to claim 10, wherein G is: 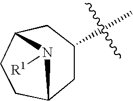
or an in vivo-hydrolysable precursor or a pharmaceutically-acceptable salt thereof.

12. A compound according to claim 10, wherein G is: 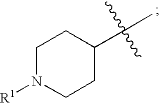
or an in vivo-hydrolysable precursor or a pharmaceutically-acceptable salt thereof.

13. A compound according to claim 10, wherein D is 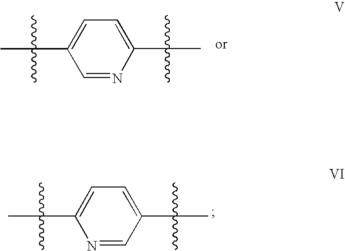
or an in vivo-hydrolysable precursor or a pharmaceutically-acceptable salt thereof.

14-17. (canceled)

18. A compound according to claim 1, wherein E is -J-Ar, J is a bond, and Ar is phenyl, phenoxy or phenyl substituted with —Cl or —O—CH₃; or an in vivo-hydrolysable precursor or a pharmaceutically-acceptable salt thereof.

19. A compound according to claim 1, wherein E is -J-Ar and J is an O.

20. A compound according to claim 1, wherein E is -J-Ar and J is a bond.

21. A compound selected from:
6-(4-Methoxy-phenyl)-N-[3-((1R,3R,5S)-8-methyl-8-aza-bicyclo[3.2.1]oct-3-yloxy)-benzyl]-nicotinamide;
N-[3-(1-Methyl-piperidin-4-yloxy)-benzyl]-6-phenyl-nicotinamide;
4-(5-{[3-((1R,3R,5S)-8-Methyl-8-aza-bicyclo[3.2.1]oct-3-yloxy)-benzylamino]-methyl}-pyridin-2-yl)-benzonitrile;
[6-(4-Methoxy-phenyl)-pyridin-3-ylmethyl]-[3-((1R,3R,5S)-8-methyl-8-aza-bicyclo[3.2.1]oct-3-yloxy)-benzyl]-amine;
[3-((1R,3R,5S)-8-Methyl-8-aza-bicyclo[3.2.1]oct-3-yloxy)-benzyl]-[6-(4-trifluoromethoxy-phenyl)-pyridin-3-ylmethyl]-amine;
[3-((1R,3R,5S)-8-Methyl-8-aza-bicyclo[3.2.1]oct-3-yloxy)-benzyl]-(6-phenyl-pyridin-3-ylmethyl)-amine;
[6-(4-Fluoro-phenyl)-pyridin-3-ylmethyl]-[3-((1R,3R,5S)-8-methyl-8-aza-bicyclo[3.2.1]oct-3-yloxy)-benzyl]-amine;
N-(3-(1-Methylpiperin-4-yloxy)benzyl)-5-(4-chlorophenyl)-N-methylisoxazole-3-carboxamide;
5-(4-Methoxy-phenyl)-isoxazole-3-carboxylic acid 3-(1-methyl-piperidin-4-yloxy)-benzylamide;
5-(4-Methoxy-phenyl)-furan-2-carboxylic acid 3-(1-methyl-piperidin-4-yloxy)-benzylamide;
5-(4-Methoxy-phenyl)-isoxazole-3-carboxylic acid 3-(8-methyl-8-aza-bicyclo[3.2.1]oct-3-yloxy)-benzylamide;
5-(4-Methoxy-phenyl)-furan-2-carboxylic acid 3-(8-methyl-8-aza-bicyclo[3.2.1]oct-3-yloxy)-benzylamide;
5-(4-Chloro-phenyl)-isoxazole-3-carboxylic acid 3-(1-methyl-piperidin-4-yloxy)-benzylamide;
5-(4-Chloro-phenyl)-isoxazole-3-carboxylic acid methyl-[3-((1R,3R,5S)-8-methyl-8-aza-bicyclo[3.2.1]oct-3-yloxy)-benzyl]-amide;
5-(4-Chloro-phenyl)-isoxazole-3-carboxylic acid 3-((1R,3R,5S)-8-methyl-8-aza-bicyclo[3.2.1]oct-3-yloxy)-benzylamide;
N-[3-((1R,3R,5S)-8-Methyl-8-aza-bicyclo[3.2.1]oct-3-yloxy)-benzyl]-6-phenoxy-nicotinamide;
5-Phenyl-2H-pyrazole-3-carboxylic acid 3-((1R,3R,5S)-8-methyl-8-aza-bicyclo[3.2.1]oct-3-yloxy)-benzylamide;
5-Phenyl-2H-pyrazole-3-carboxylic acid 3-(1-methyl-piperidin-4-yloxy)-benzylamide;
6-Chloro-N-[3-((1R,3R,5S)-8-methyl-8-aza-bicyclo[3.2.1]oct-3-yloxy)-benzyl]-nicotinamide;
[3-(1-Methyl-piperidin-4-yloxy)-benzyl]-(6-phenoxy-pyridin-3-ylmethyl)-amine;
[5-(4-Fluoro-phenyl)-isoxazol-3-ylmethyl]-[3-((1R,3R,5S)-8-methyl-8-aza-bicyclo[3.2.1]oct-3-yloxy)-benzyl]-amine;
[3-((1R,3R,5S)-8-Methyl-8-aza-bicyclo[3.2.1]oct-3-yloxy)-benzyl]-(6-phenoxy-pyridin-3-ylmethyl)-amine;
[5-(4-Chloro-phenyl)-isoxazol-3-ylmethyl]-methyl-[3-((1R,3R,5S)-8-methyl-8-aza-bicyclo[3.2.1]oct-3-yloxy)-benzyl]-amine;
5-(3-Methoxyphenyl)pyridine-2-carboxylic acid 3-((1R,3R,5S)-8-methyl-8-aza-bicyclo[3.2.1]oct-3-yloxy)benzylamide;
N-(3-(1-Methylpiperidin-4-yloxy)benzyl)-5-(4-methoxyphenyl)pyridine-2-carboxamide;
N-(3-(1-Methylpiperidin-4-yloxy)benzyl)-5-phenylpyridine-2-carboxamide;
5-(4-Methoxyphenyl)pyridine-2-carboxylic acid 3-((1R,3R,5S)-8-methyl-8-aza-bicyclo[3.2.1]oct-3-yloxy)benzylamide;
5-(3-Methoxyphenyl)pyridine-2-carboxylic acid 3-(1-methylpiperidin-4-yloxy)benzylamide;
5-Phenylpyridine-2-carboxylic acid 3-((1R,3R,5S)-8-methyl-8-aza-bicyclo[3.2.1]oct-3-yloxy)benzylamide; and
5-(4-Methoxy-phenyl)-[1,2,4]oxadiazole-3-carboxylic acid 3-((1R,3R,5S)-8-methyl-8-aza-bicyclo[3.2.1]oct-3-yloxy)-benzylamide;
or an in vivo-hydrolysable precursor or a pharmaceutically-acceptable salt of any foregoing compound.

22. A method of treatment or prophylaxis of a disease or condition in which modulation of the MCH1 receptor is beneficial comprising administering to a subject suffering from said disease or condition a therapeutically-effective amount of a compound in accord with Formula I: 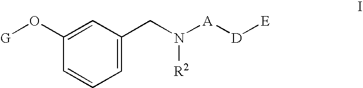
wherein:
G is: 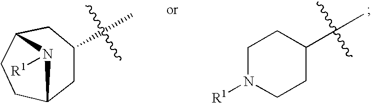
R¹ is H. —C₁₋₆alkyl, C₁₋₆haloalkyl, —C₃₋₈cycloalkyl, or —C₃₋₈cyclooxyalkyl;
R² is hydrogen or —C₁₋₄alkyl;
A is —CH₂— or —C(═O)—,
D is a 5- or 6-membered aromatic heterocyclic moiety having 1, 2 or 3 heteroatoms selected from nitrogen, oxygen or sulfur where not more than one of said heteroatoms is oxygen or sulfur, or an 8-, 9- or 10-membered fused aromatic heterocyclic moiety having 1, 2 or 3 heteroatoms selected from nitrogen, oxygen or sulfur where not more than one of said heteroatoms is oxygen or sulfur;
E is a hydrogen, halogen, —CN, —NO₂, —CF₃, —CONR⁷R⁸, —S(O)ₙR⁷, —NR⁷R⁸, —CH₂NR⁷R⁸, —OR⁷, —CH₂OR⁷, —NC(═O)R⁷, —CO₂R⁷, —C₁₋₆alkyl —C₂₋₆alkenyl —C₂₋₆alkynyl, C₁₋₆alkoxy, C₃₋₈cycloalkyl —O—CH₂—O— or -J-Ar;
J is —O—, —CH₂—, —O—CH₂— or a bond;
Ar is a 5- or 6-membered aromatic or heteroaromatic moiety having 0, 1 or 2 nitrogen atoms, 0 or 1 oxygen atoms, and 0 or 1 sulfur atoms, or an 8-, 9- or 10-membered fused aromatic or heteroaromatic ring system having 0, 1, 2 or 3 nitrogen atoms, 0 or 1 oxygen atoms, and 0 or 1 sulfur atoms;
wherein Ar is unsubstituted or has 1, 2 or 3 substituents independently selected at each occurrence from —C₁₋₆alkyl, —C₂₋₆alkenyl, —C₂₋₆alkynyl, halogen, —CN, —NO₂, —CF₃, —CONR⁷R⁸, —S(O)ₙR⁷, —NR⁷R⁸, —CH₂NR⁷R⁸, —OR⁷, —CH₂OR⁷, —NC(═O)R⁷, and —CO₂R⁷;
n is 1, 2, or 3; and
R⁷ and R⁸ are each independently hydrogen, CF₃, C₁₋₆alkyl and/or C₃₋₈cycloalkyl;
or in vivo-hydrolysable precursors or pharmaceutically-acceptable salts thereof.

23. The method of claim 22, wherein said disease or condition is mood changes, anxiety, depression, generalized anxiety disorder, panic attacks, panic disorder, obsessive-compulsive disorder, bipolar disorder, obesity and related disorders, eating disorders, psychiatric disorders, neurological disorders, and pain.

24-25. (canceled)

26. A method of treatment or prophylaxis of mood changes, anxiety, depression, generalized anxiety disorder, panic attacks, panic disorder, obsessive-compulsive disorder, bipolar disorder, obesity and related disorders, eating disorders, psychiatric disorders, neurological disorders, and pain-comprising administering to a patient suffering therefrom a therapeutically-effective amount of at least one compound according to claim 1.

27. (canceled)

28. A pharmaceutical composition comprising at least one pharmaceutically-acceptable diluent, lubricant, and/or carrier and at least one compound according to claim 1.

29. A method of treatment or prophylaxis of a disease or condition in which modulation of the MCH1 receptor is beneficial comprising administering a therapeutically-effective amount of a pharmaceutical composition according to claim 28 to a patient suffering from said disease or condition.

30. The method of claim 29, wherein said disease or condition is mood changes, anxiety, depression, generalized anxiety disorder, panic attacks, panic disorder, obsessive-compulsive disorder, bipolar disorder, obesity and related disorders, eating disorders, psychiatric disorders, neurological disorders, and pain.

31-41. (canceled)
